(19) United States
(12) Patent Application Publication
(10) Pub. No.: US 2025/0387046 A1
(43) Pub. Date: Dec. 25, 2025

(54) SYSTEM, PATIENT SUPPORT APPARATUS AND METHOD FOR DETERMINING IMMOBILITY OF A PATIENT

(71) Applicant: UMANO MEDICAL INC., L'Islet (CA)

(72) Inventors: David Mayen MORENO, L'Ancienne-Lorette (CA); Anouer KEBIR, Lévis (CA); Esther BERTHELOT, Lévis (CA); Steve BOLDUC, Beaumont (CA); Carine HUET, Rivedoux-Plage (FR); Luc LANDRY, La Pocatière (CA); Rudy BÉLANGER, La Pocatière (CA); Geneviève CARON, La Pocatière (CA); Stéphane DOYON, La Pocatière (CA); Charles FORTIER, Lévis (CA)

(21) Appl. No.: 19/237,815

(22) Filed: Jun. 13, 2025

Related U.S. Application Data
(63) Continuation-in-part of application No. 19/137,565, filed on Jan. 1, 0001.
(60) Provisional application No. 63/434,379, filed on Dec. 21, 2022.

(51) Int. Cl. A61B 5/11; A61B 5/00; A61B 5/103; A61G 7/05

(52) U.S. Cl. CPC A61B 5/1118; A61B 5/1036; A61B 5/4815; A61B 5/6892; A61B 5/7278; A61B 5/7282; A61B 5/742; A61G 7/0527; A61B 2562/0252

(57) ABSTRACT

There is provided a system, a patient support apparatus and a method for determining immobility of a patient based on a level of patient activity. The patient support apparatus has at least one load cell. Sensor data from the at least one load cell is used to compute the level of patient activity. If the level of patient activity is below a threshold, the movement of the center of gravity of the patient is computed. If the movement of the center of gravity is below a movement threshold, the patient is determined to be immobile. An indication of immobility associated with an immobility time period is displayed.

### CROSS-REFERENCE TO RELATED APPLICATIONS

The present application is continuation-in-part of U.S. patent application Ser. No. 19/137,565 filed on Jun. 10, 2025, which is a national stage entry application under 35 U.S.C. § 371 of International Patent Application No. PCT/IB2023/063114 filed on Dec. 21, 2023, which claims priority from U.S. Provisional Patent Application Ser. No. 63/434,379 filed on Dec. 21, 2022, each of which is incorporated by reference herein.

### TECHNICAL FIELD

The present technology relates to patient activity detection, and in particular to methods, systems and patient support apparatuses configured for determining immobility of a patient in patient support apparatus.

### BACKGROUND

Patient activity level is an indicator of health and has been shown to predict clinical outcomes. However, in a hospital setting, patient activity status is not convenient to measure. Conventionally, patient activity level is determined by using metrics such as the Cohen-Mansfield Agitation Inventory (CMAI), which requires a caregiver to record observations of the patient over a period of two weeks.

It can be beneficial to monitor the general activity level of a patient on a shorter timescale and without continual observation of the patient by a caregiver, particularly in a hospital environment.

There is a desire for additional methods of determining a level of patient activity.

### SUMMARY

There is disclosed a system and method of determining an activity level of a patient in a patient support apparatus such as a hospital bed by using the load cells of the patient support apparatus.

There is disclosed a system and method of determining an activity level of a patient in a patient support apparatus such as a hospital bed based on the total amount of weight detected in the hospital bed.

There is disclosed a system and method of determining an activity level of a patient in a hospital bed based on a rate of change in the total amount of weight detected in the hospital bed.

There is disclosed a system and method of determining an activity level of a patient in a patient support apparatus such as a hospital bed that is not invasive or intrusive.

In accordance with a broad aspect of the present technology, there is provided a system for determining a level of activity of a patient in a patient support apparatus having at least one load cell. The system comprises a non-transitory storage medium storing computer-readable instructions thereon, and at least one processor operatively connected to the non-transitory storage medium and to the at least one load cell. The at least one processor, upon executing the computer-readable instructions is configured for: determining a total weight detected by the at least one load cell, determining changes in the total weight detected by the at least one load cell over a given period of time, determining an operational characteristic of the changes in the total weight based on at least one of a magnitude and a time duration of the changes in total weight, and determining a level of activity of the patient during the given period of time based on the operational characteristic.

In one or more implementations of the system, the operational characteristic comprises a rate of change of the total weight detected by the at least one load cell.

In one or more implementations of the system, the operational characteristic comprises a duration of a change of the total weight detected by the at least one load cell.

In one or more implementations of the system, the operational characteristic comprises a variance of the total weight detected by the at least one load cell over a predetermined period of time.

In one or more implementations of the system, the operational characteristic comprises a range of the total weight detected by the at least one load cell over a predetermined period of time.

In one or more implementations of the system, the operational characteristic comprises a square of the range of the total weight detected by the at least one load cell over the predetermined period of time.

In one or more implementations of the system, determining the operational characteristic comprises: comparing the total weight to a first threshold and determining that the total weight detected by the at least one load cell has increased beyond the first threshold.

In one or more implementations of the system, determining the operational characteristic comprises determining that the total weight detected by the at least one load cell has increased above a first threshold weight.

In one or more implementations of the system, determining the operational characteristic comprises determining that the total weight detected by the at least one load cell has decreased below a second threshold weight.

In one or more implementations of the system, determining the operational characteristic comprises comparing the total weight detected by the at least one load cell to a moving average of the total weight detected by the at least one load cell.

In one or more implementations of the system, the at least one processor is operatively connected to a display screen, and the at least one processor is further configured to cause display of an indication of the level of activity of the patient on the display screen.

In one or more implementations of the system, the indication comprises a notification that the level of activity of the patient is below a first threshold level of activity.

In one or more implementations of the system, the indication comprises a notification that the level of activity of the patient is above a second threshold level of activity.

In one or more implementations of the system, said determining the operational characteristic comprises normalizing the change in the total weight by a weight of the patient.

In one or more implementations of the system, the at least one load cell is a plurality of load cells.

In one or more implementations of the system, said determining changes in the total weight detected by the at least one load cell over the given period of time comprises filtering at least a portion of data detected by the at least one load cell over the given period of time to determine the changes in the total weight.

In one or more implementations of the system, the level of activity is on a scale from 0 to 4.

In one or more implementations of the system, the at least one processor is further configured for: determining a presence of the patient in the patient support apparatus based on the determined changes in total weight.

In one or more implementations of the system, the at least one processor is further configured for: determining a time spent by the patient in the patient support apparatus based on the determined changes in total weight.

In one or more implementations of the system, the at least one processor is further configured for: causing display of an indication of the time spent by the patient in the patient support apparatus.

In one or more implementations of the system, the at least one processor is further configured for: determining a further operational characteristic based on the total weight detected by the at least one load cell, comparing the further operational characteristic to a threshold, and if the further operational characteristic is one of equal to and above the threshold: determining that the level of activity is potentially influenced by an external motion, and transmitting an indication that the level of activity is potentially influenced by the external motion.

In one or more implementations of the system, the further operational characteristic comprises a variance of the total weight over another given period of time, the another given period of time being one of: equal to or less than the given period of time.

In accordance with a broad aspect of the present technology, there is provided a system of monitoring an activity level of a patient in a patient support apparatus, the patient support apparatus having at least one load cell. The system comprises a non-transitory storage medium storing computer-readable instructions thereon, at least one processor operatively connected to the non-transitory storage medium and to the at least one load cell, and a display screen operatively connected to the at least one processor. The at least one processor, upon executing the computer-readable instructions, being configured for: monitoring a position of a center of mass of the patient via at the least one load cell, monitoring an activity level of the patient via the at least one load cell, and displaying to a user on the display screen a current position of the center of mass of the patient in the patient support apparatus, with an indication corresponding to a current activity level of the patient.

In one or more implementations of the system, the at least one processor is further configured for: displaying to the user on the display screen a past position of the center of mass of the patient in the patient support apparatus, with an indication corresponding to a corresponding past activity level of the patient.

In one or more implementations of the system, displaying comprises displaying on a plurality of display screens.

In one or more implementations of the system, the display screen is disposed on the patient support apparatus.

In one or more implementations of the system, the display screen is disposed on an electronic device remote from the patient support apparatus.

In one or more implementations of the system, displaying comprises displaying a video image.

In one or more implementations of the system, displaying comprises displaying on a printed report.

In one or more implementations of the system, the at least one load cell is a plurality of load cells.

In accordance with a broad aspect of the present technology, there is provided a system of determining a presence of a patient in a patient support apparatus, the patient support apparatus having at least one load cell. The system comprises: a non-transitory storage medium storing computer-readable instructions thereon, and at least one processor operatively connected to the non-transitory storage medium and to the at least one load cell. The at least one processor, upon executing the computer-readable instructions, is configured for: determining a weight associated with at least a portion of the patient support apparatus over a given period of time, using the at least one load cell, determining a level of activity associated with the portion of the patient support apparatus over the given period of time, using the at least one load cell, and determining a presence of the patient in the patient support apparatus based on the determined weight over the given period of time.

In one or more implementations of the system, the at least one load cell is a plurality of load cells.

In one or more implementations, the at least one processor is further configured for determining a time spent by the patient in the patient support apparatus based on the determined weight and the determined level of activity.

In one or more implementations, the at least one processor is further configured for causing display of an indication of at least one of: the level of activity, the presence of the patient in the patient support apparatus and the time spent by the patient in the patient support apparatus.

In accordance with another broad aspect of the present technology, there is provided a system of predicting an activity level of a patient. The system comprises a non-transitory storage medium storing computer-readable instructions thereon, and at least one processor operatively connected to the non-transitory storage medium and to the at least one load cell. The at least one processor, upon executing the computer-readable instructions, is configured for: determining a weight associated with at least a portion of the patient support apparatus, using at least one load cell, determining a level of activity associated with the portion of the patient support apparatus, using the at least one load cell, and predicting a future activity level of the patient based on the determined weight and the determined level of activity.

In one or more implementations of the system, predicting the future activity level of the patient comprises predicting a probability of aggressive behavior by the patient.

In one or more implementations of the system, predicting a future activity level of the patient comprises predicting that an activity level of the patient will be higher or lower than a recommended activity level for the patient.

In one or more implementations of the system, the at least one load cell is a plurality of load cells.

In one or more implementations of the system, the at least one processor has access to at least one trained machine learning (ML) model, and said predicting the future activity level of the patient based on the determined weight and the determined level of activity comprises using the at least one trained ML model.

In accordance with another broad aspect of the present technology, there is provided a patient support apparatus comprising: at least one load cell, at least one processor, and a non-transitory memory connected to the at least one processor, the non-transitory memory including computer-readable instructions that, when executed, cause the at least one processor to: determine a total weight detected by the at least one load cell, determine changes in the total weight detected by the at least one load cell over a given period of time, determine an operational characteristic of the changes in the total weight based on at least one of a magnitude and a time duration of the changes in total weight, and determine a level of activity of the patient during the given period of time based on the operational characteristic.

In one or more implementations of the patient support apparatus, the at least one load cell is a plurality of load cells.

In accordance with another broad aspect of the present technology, there is provided a system comprising: a patient support apparatus having at least one load cell, an electronic device connectable to the patient support apparatus via a communications network, the electronic device comprising: at least one processor, and a non-transitory memory connected to the at least one processor, the non-transitory memory including computer-readable instructions that, when executed, cause the at least one processor to: receive, from the patient support apparatus, data indicative of a total weight detected by the at least one load cell, determine changes in the total weight detected by the at least one load cell over a given period of time, determine an operational characteristic of the changes in the total weight based on at least one of a magnitude and a time duration of the changes in total weight, and determine a level of activity of the patient during the given period of time based on the operational characteristic.

In one or more implementations of the system, the at least one load cell is a plurality of load cells.

According to a broad aspect, there is provided a method of determining a level of activity of a patient in a patient support apparatus, the patient support apparatus having at least one load cell, the method comprising: determining a total weight detected by the at least one load cell; determining changes in the total weight detected by the at least one load cell over a given period of time; determining an operational characteristic of the changes in the total weight based on at least one of a magnitude and a time duration of the changes in total weight; and determining a level of activity of the patient during the given period of time based on the operational characteristic.

Optionally, in any of the previous aspects, the operational characteristic comprises a rate of change of the total weight detected by the at least one load cell.

Optionally, in any of the previous aspects, the operational characteristic comprises a duration of a change of the total weight detected by the at least one load cell.

Optionally, in any of the previous aspects, the operational characteristic comprises a variance of the total weight detected by the at least one load cell over a predetermined period of time.

Optionally, in any of the previous aspects, the operational characteristic comprises a range of the total weight detected by the at least one loadcells over a predetermined period of time.

Optionally, in any of the previous aspects, the operational characteristic comprises a square of the range of the total weight detected by the at least one load cell over the predetermined period of time.

Optionally, in any of the previous aspects, determining the operational characteristic comprises determining that the total weight detected by the at least one load cell has increased beyond a first threshold.

Optionally, in any of the previous aspects, determining the operational characteristic comprises determining that the total weight detected by the at least one load cell has increased above a first threshold weight.

Optionally, in any of the previous aspects, determining the operational characteristic comprises determining that the total weight detected by the at least one load cell has decreased below a second threshold weight.

Optionally, in any of the previous aspects, determining the operational characteristic comprises comparing the total weight detected by the at least one load cell to a moving average of the total weight detected by the at least one load cell.

Optionally, in any of the previous aspects, the method includes displaying an indication of the level of activity of the patient.

Optionally, in any of the previous aspects, the indication comprises a notification that the level of activity of the patient is below a first threshold level of activity.

Optionally, in any of the previous aspects, the indication comprises a notification that the level of activity of the patient is above a second threshold level of activity.

Optionally, in any of the previous aspects, determining the operational characteristic comprises normalizing the change in the total weight by a weight of the patient.

Optionally, in any of the previous aspects, the at least one load cell is a plurality of load cells.

Optionally, in any of the previous aspects, said determining changes in the total weight detected by the at least one load cell over the given period of time comprises filtering at least a portion of data detected by the at least one load cell over the given period of time to determine the changes in the total weight.

Optionally, in any of the previous aspects, the level of activity is on a scale from 0 to 4.

Optionally, in any of the previous aspects, the method further comprises determining a presence of the patient in the patient support apparatus based on the determined change in total weight.

Optionally, in any of the previous aspects, the method further comprises: determining a time spent by the patient in the patient support apparatus based on the determined change in total weight.

Optionally, in any of the previous aspects, the method further comprises: causing display of an indication of the time spent by the patient in the patient support apparatus.

Optionally, in any of the previous aspects, the method further comprises determining a further operational characteristic based on the total weight detected by the at least one load cell, comparing the further operational characteristic to a threshold, and if the further operational characteristic is one of equal to and above the threshold: determining that the level of activity is potentially influenced by an external motion, and transmitting an indication that the level of activity is potentially influenced by the external motion.

Optionally, in any of the previous aspects, the further operational characteristic comprises a variance of the total weight over another given period of time, the another given period of time being one of: equal to or less than the given period of time.

According to another broad aspect, there is provided a method of monitoring an activity level of a patient in a patient support apparatus, the patient support apparatus having at least one load cell, the method comprising: monitoring a position of a center of mass of the patient via at least one load cell; monitoring an activity level of the patient via the at least one load cell; and displaying to a user a current position of the center of mass of the patient in the patient support apparatus, with an indication corresponding to a current activity level of the patient.

Optionally, in any of the previous aspects, the method includes displaying to the user a past position of the center of mass of the patient in the patient support apparatus, with an indication corresponding to a corresponding past activity level of the patient.

Optionally, in any of the previous aspects, displaying comprises displaying on a screen.

Optionally, in any of the previous aspects, the screen is disposed on the patient support apparatus.

Optionally, in any of the previous aspects, the screen is disposed on an electronic device remote from the patient support apparatus.

Optionally, in any of the previous aspects, displaying comprises displaying a video image.

Optionally, in any of the previous aspects, displaying comprises displaying on a printed report.

Optionally, in any of the previous aspects, the at least one load cell is a plurality of load cells.

According to another broad aspect, there is provided a method of determining a presence of a patient in a patient support apparatus, the patient support apparatus having at least one load cell, the method comprising: determining a weight associated with at least a portion of the patient support apparatus over a given period of time, using the at least one load cell; determining a level of activity associated with the portion of the patient support apparatus, using the at least one load cell, and determining a presence of the patient in the patient support apparatus over the given period of time based on the determined weight.

In one or more implementations, the method further comprises determining a time spent by the patient in the patient support apparatus based on the determined weight over the given period of time.

In one or more implementations, the method further comprises causing display of an indication of at least one of: the level of activity, the presence of the patient in the patient support apparatus and the time spent by the patient in the patient support apparatus.

Optionally, in any of the previous aspects, the at least one load cell is a plurality of load cells.

According to another broad aspect, there is provided a method of predicting an activity level of a patient, comprising: determining a weight associated with at least a portion of the patient support apparatus, using at least one load cell; determining a level of activity associated with the portion of the patient support apparatus, using the at least one load cell; and predicting a future activity level of the patient based on the determined weight and the determined level of activity.

Optionally, in any of the previous aspects, predicting the future activity level of the patient comprises predicting a probability of aggressive behavior by the patient.

Optionally, in any of the previous aspects, predicting a future activity level of the patient comprises predicting that an activity level of the patient will be higher or lower than a recommended activity level for the patient.

Optionally, in any of the previous aspects, the at least one load cell is a plurality of load cells.

In accordance with a broad aspect of the present technology, there is provided a system for determining immobility of a patient in a patient support apparatus, the system comprising: a non-transitory storage medium storing computer-readable instructions, at least one processor operatively connected to the non-transitory storage medium, the at least one processor being operatively connected to at least one load cell of the patient support apparatus. The at least one processor, upon executing the computer-readable instructions, is configured for: receiving, from the at least one load cell, sensor data comprising weight components exerted by the patient, computing, based on the sensor data, a level of patient activity of the patient without calculating a center of gravity of the patient, determining if the level of patient activity is below a predetermined threshold during a given period of time, if the level of patient activity is below the predetermined threshold: computing a movement of the center of gravity of the patient during the given period of time, determining if the movement of the center of gravity of the patient is below a movement threshold, if the movement of the center of gravity is below the movement threshold: determining that the patient is immobile during the given period of time, and transmitting, to a user interface operatively connected to the at least one processor, an indication of immobility of the patient during the given period of time.

In one or more embodiments of the system, the at least one processor is further configured for, prior to said computing the level of patient activity of the patient: computing, based on the sensor data comprising the weight components, a total weight detected by the at least one load cell, computing changes in the total weight detected by the at least one load cell over the given period of time, computing an operational characteristic of the changes in the total weight based on at least one of a magnitude and a time duration of the changes in total weight, and said determining the level of patient activity of the patient is based on the operational characteristic.

In one or more embodiments of the system, said computing a level of patient activity of the patient during the given period of time based on the operational characteristic comprises: computing a variance of the changes in total weight over the given period of time, and linearizing the variance of the weight to obtain the level of patient activity of the patient.

In one or more embodiments of the system, said computing the movement of the center of gravity of the patient comprises: computing a difference between consecutive positions of the center of gravity of the patient during the given period of time.

In one or more embodiments of the system, the user interface comprises a display operatively connected to the at least one processor, and the at least one processor is further configured for displaying the indication of immobility and the given time period on a display operatively connected to the at least one processor.

In one or more embodiments of the system, the at least one processor is configured for displaying the indication of immobility and the given time period in a timeline.

In one or more embodiments of the system, the at least one processor is further configured for: receiving, from the user interface, a maximum time of immobility, determining if the given period of time is equal to above the maximum time of immobility, and if the given period of time is equal to or above the maximum time of immobility: transmitting an indication that the patient has been immobile above the maximum time.

In one or more embodiments of the system, said transmitting indication that the patient has been immobile above the maximum time causes generation of at least one of: a visual alert and an audio alert.

In one or more embodiments of the system, the level of patient activity varies between 0 and 4.

In one or more embodiments of the system, the predetermined threshold is equal to 2.5.

In accordance with a broad aspect of the present technology, there is provided a patient support apparatus comprising: a frame, at patient support surface supported by the frame, at least two load cells disposed between the frame and the patient support surface, and at least one processor operatively connected to the at least one two cells, the at least one processor being configured for: receiving, from the at least two load cells, sensor data comprising weight components exerted by the patient, computing, based on the sensor data, a level of patient activity of the patient, determining if the level of patient activity is below a predetermined threshold during a given period of time, if the level of patient activity is below the predetermined threshold: computing a movement of a center of gravity of the patient during the given period of time, determining if the movement of the center of gravity of the patient is below a movement threshold, if the movement of the center of gravity is below the movement threshold: determining that the patient is immobile during the given period of time, and transmitting, to a user interface operatively connected to the at least one processor, an indication of immobility of the patient during the given period of time.

In one or more embodiments of the patient support apparatus, the patient support apparatus the at least two load cells comprise four load cells.

In accordance with a broad aspect of the present technology, there is provided a method for determining immobility of a patient supported by a patient support apparatus, the patient support apparatus having at least one load cell, the method being executed by at least one processor operatively connected to the at least one load cell. The method comprises: receiving, from the at least one load cell, sensor data comprising weight components exerted by the patient, computing, based on the sensor data, a level of patient activity of the patient without computing a center of gravity of the patient, determining if the level of patient activity is below a predetermined threshold during a given period of time, if the level of patient activity is below the predetermined threshold: computing a movement of the center of gravity of the patient during the given period of time, determining if the movement of the center of gravity of the patient is below a movement threshold, if the movement of the center of gravity is below the movement threshold: determining that the patient is immobile during the given period of time, and transmitting, to a user interface operatively connected to the at least one processor, an indication of immobility of the patient during the given period of time.

In one or more embodiments of the method, the method further comprises, prior to said computing the level of patient activity of the patient: computing, based on the sensor data comprising the weight components, a total weight detected by the at least one load cell, computing changes in the total weight detected by the at least one load cell over the given period of time, computing an operational characteristic of the changes in the total weight based on at least one of a magnitude and a time duration of the changes in total weight, and said determining the level of patient activity of the patient is based on the operational characteristic.

In one or more embodiments of the method, said computing a level of patient activity of the patient during the given period of time based on the operational characteristic comprises: computing a variance of the changes in total weight over the given period of time, and linearizing the variance of the weight to obtain the level of patient activity of the patient.

In one or more embodiments of the method, said computing the movement of the center of gravity of the patient comprises: computing a difference between consecutive positions of the center of gravity of the patient during the given period of time.

In one or more embodiments of the method, the method further comprises: displaying the indication of immobility and the given time period on a display operatively connected to the at least one processor.

In one or more embodiments of the method, the method further comprises: receiving, from the user interface, a maximum time of immobility, determining if the given period of time is above the maximum time of immobility, and if the given period of time is equal to or above the maximum time of immobility: transmitting an indication that the patient has been immobile above the maximum time.

In one or more embodiments of the method, said transmitting indication that the patient has been immobile above the maximum time causes generation of at least one of: a visual alert and an audio alert.

In one or more embodiments of the method, the level of patient activity varies between 0 and 4.

In one or more embodiments of the method, the predetermined threshold is equal to 2.5.

### BRIEF DESCRIPTION OF THE DRA WINGS

Having thus generally described the nature of the present technology, reference will now be made to the accompanying drawings, showing by way of illustration example implementations thereof and in which:

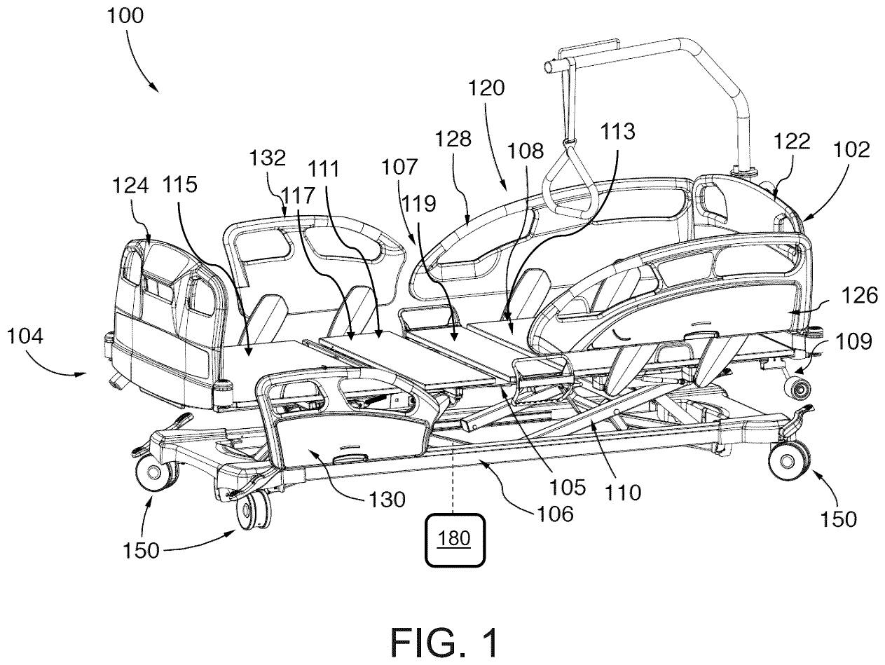

FIG. 1 illustrates a perspective view of a hospital bed to which the present disclosure can be applied;

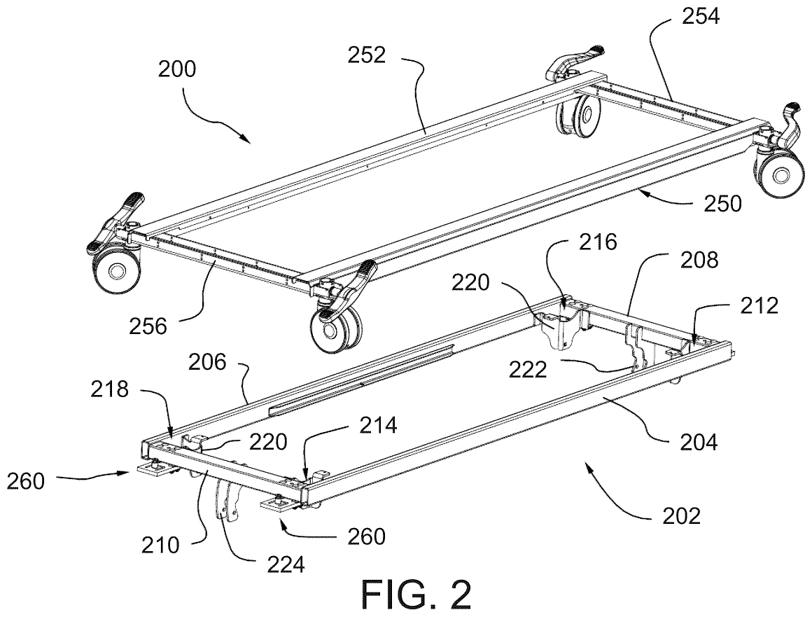

FIG. 2 illustrates an exploded perspective view of a base portion of the bed of FIG. 1;

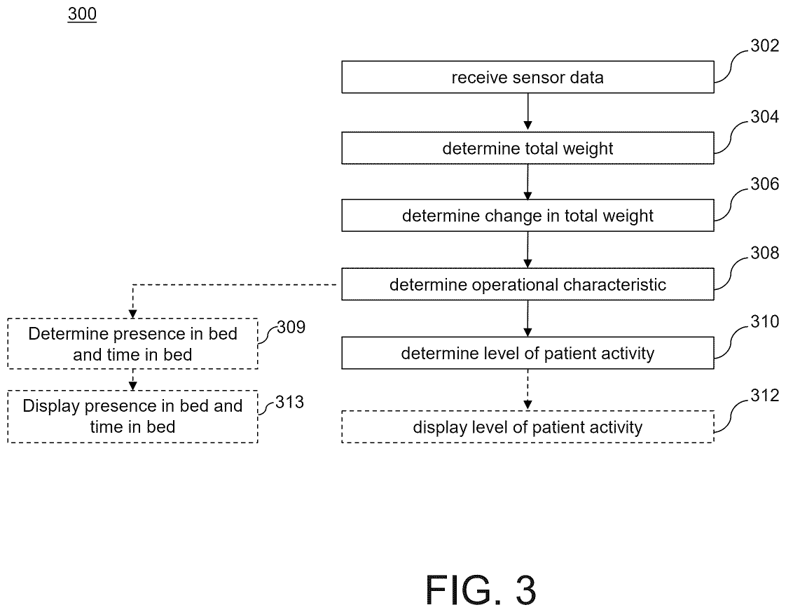

FIG. 3 illustrates a flowchart of a method of detecting patient activity according to one or more implementations;

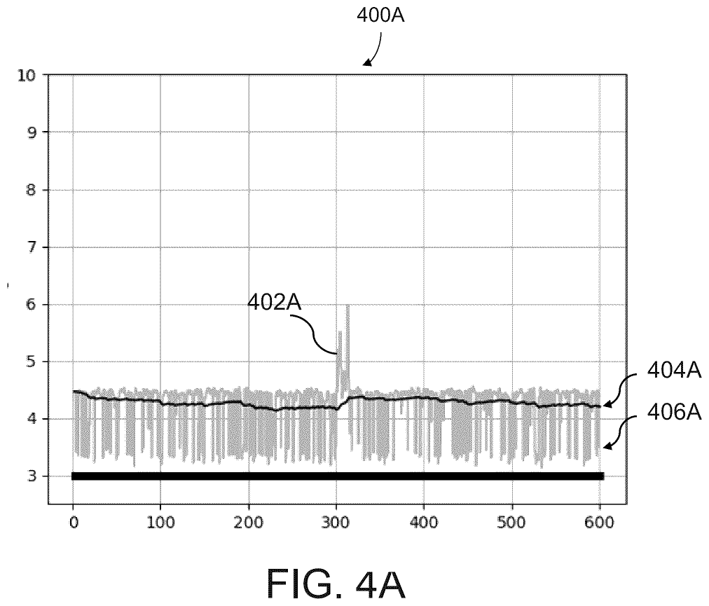

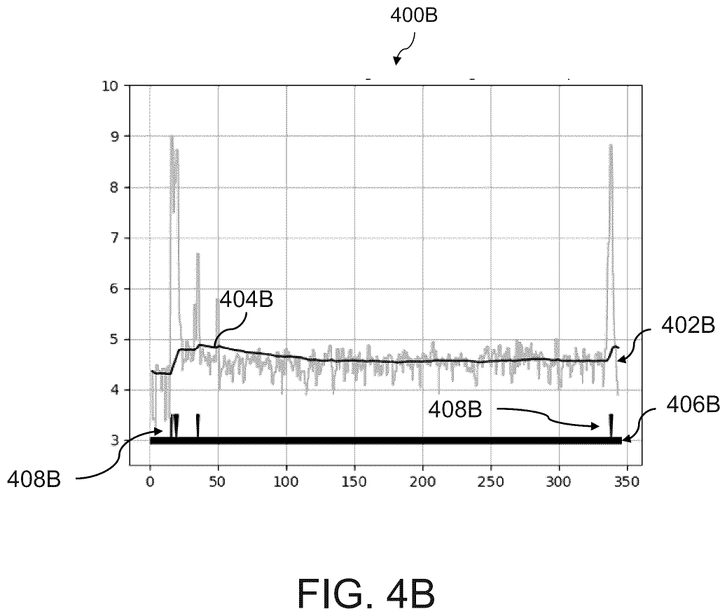

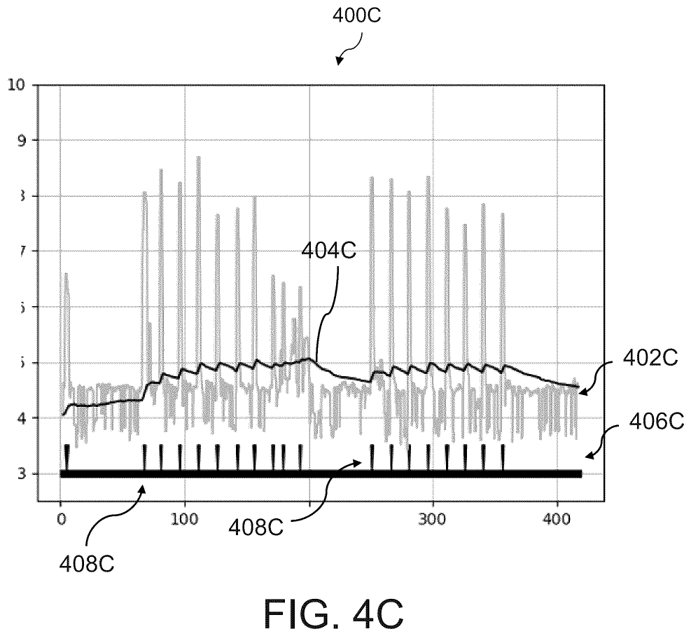

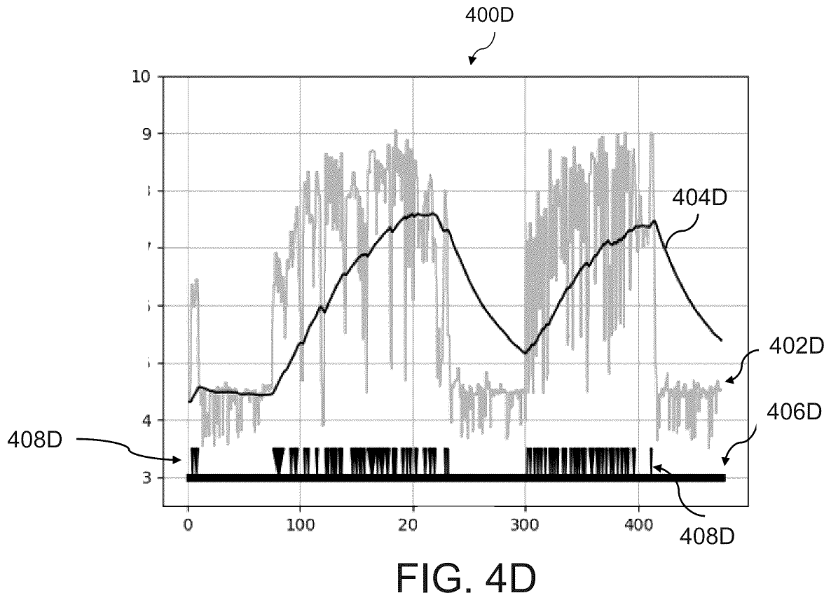

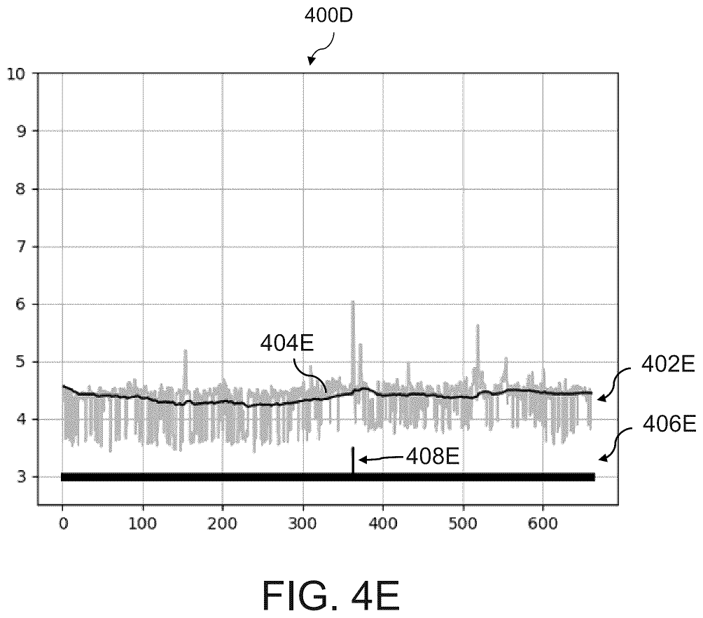

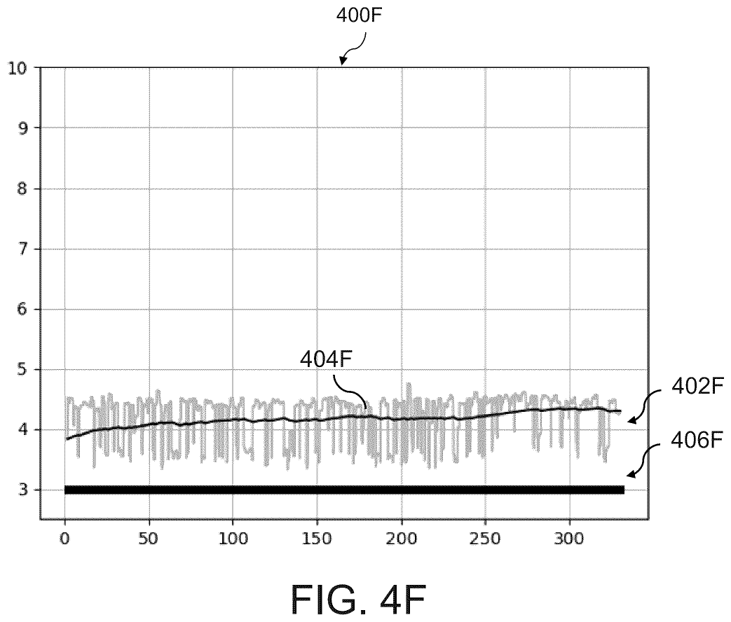

FIG. 4A illustrates a non-limiting example of a graph showing an activity level when the bed is not occupied by a patient;

FIG. 4B illustrates a non-limiting example of a graph showing an activity level when the bed is occupied by a calm patient with the patient entering and leaving the hospital bed;

FIG. 4C illustrates a non-limiting example of a graph showing an activity level when the bed is occupied by a patient kicking the bed;

FIG. 4D illustrates a non-limiting example of a graph showing an activity level when the bed is occupied by a patient showing convulsions;

FIG. 4E illustrates a non-limiting example of a graph showing an activity level when the bed is occupied by a patient moving his hand occasionally;

FIG. 4F illustrates a non-limiting example of a graph showing an activity level when a person walks near the hospital bed;

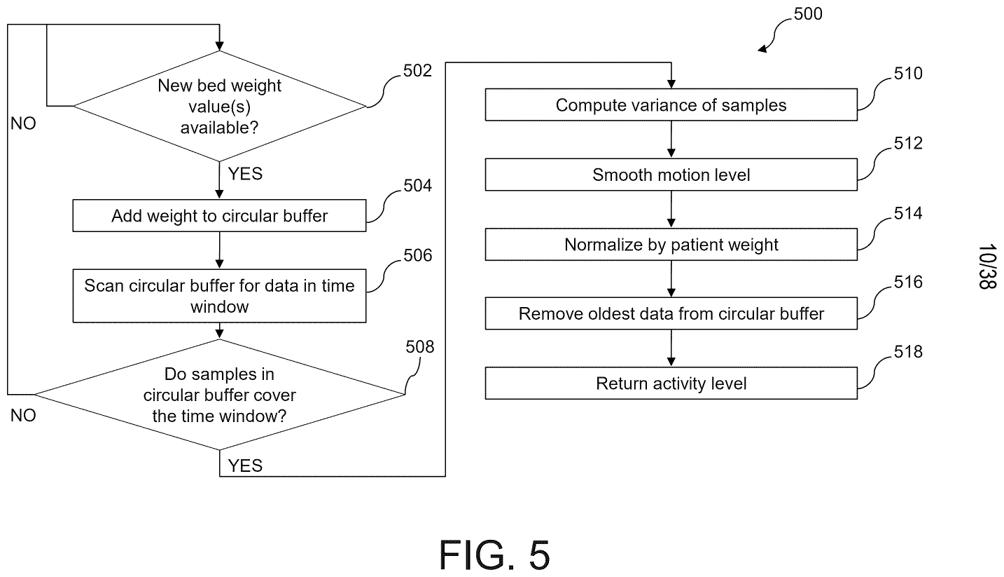

FIG. 5 illustrates a flowchart of a method of processing weight sensor data of a hospital bed in accordance with one or more non-limiting implementations of the present technology;

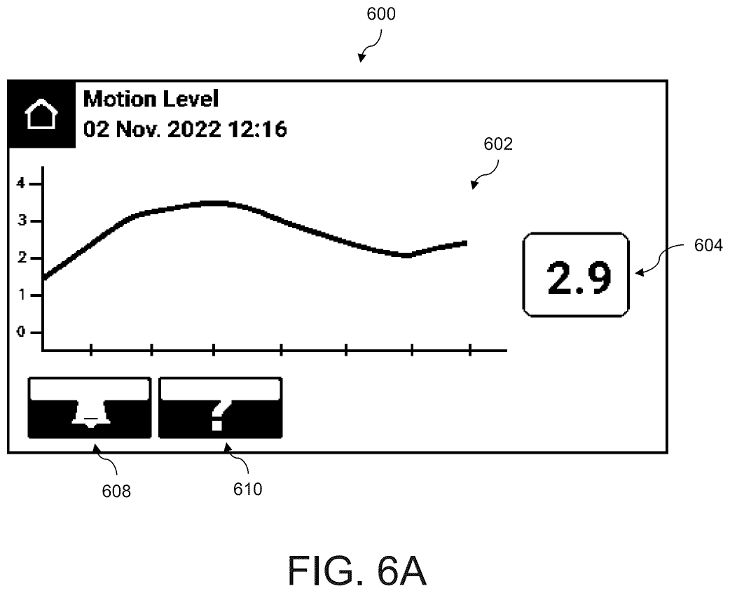

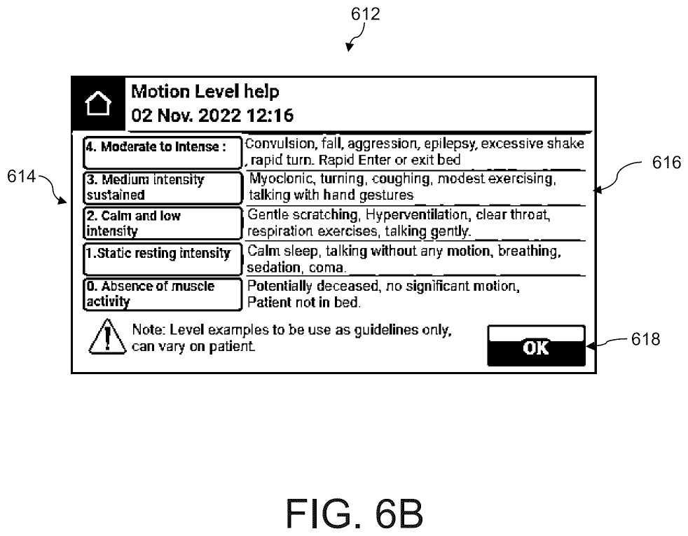

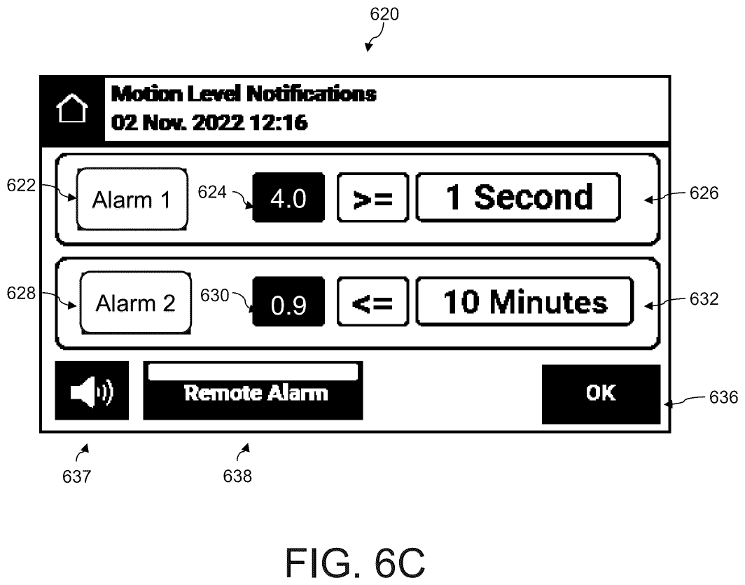

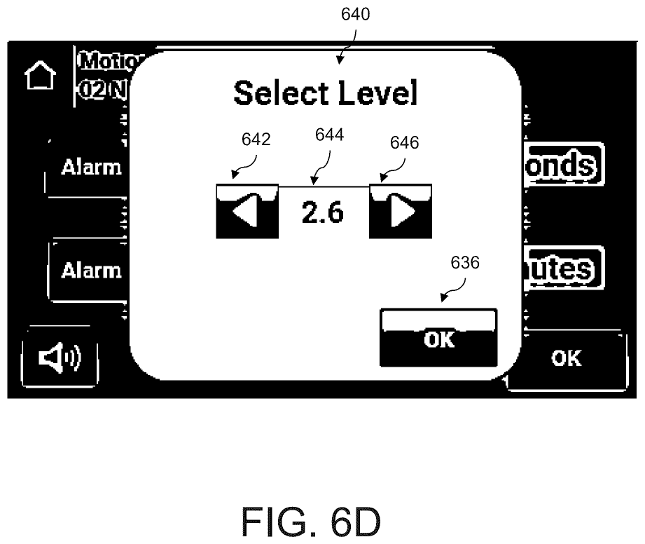

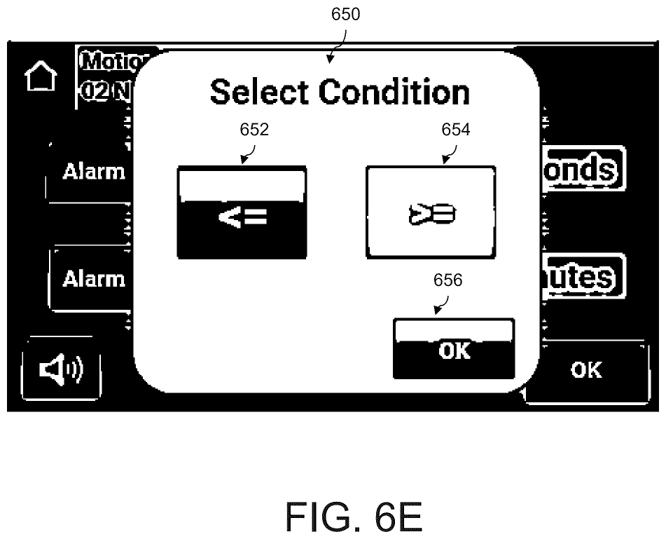

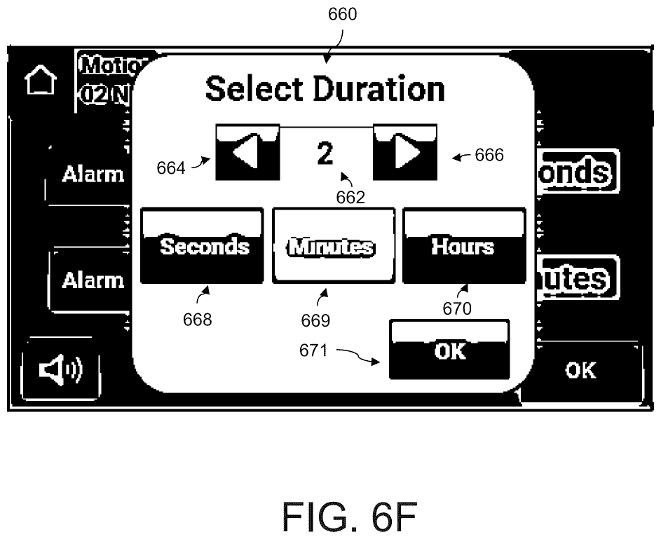

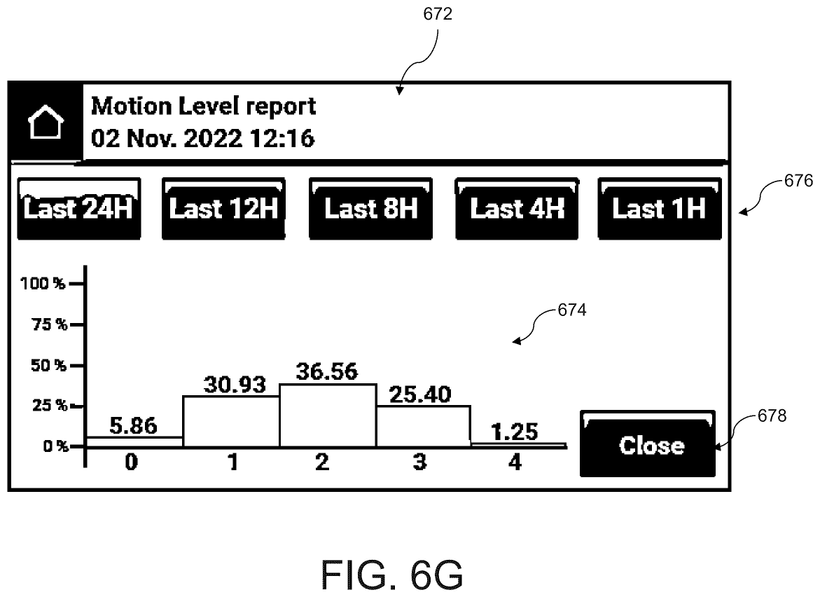

FIG. 6A illustrates a non-limiting example of a patient activity graphical user interface (GUI);

FIG. 6B illustrates a non-limiting example of a help GUI displayed by selecting a help button in the patient activity GUI of FIG. 6A;

FIG. 6C illustrates a non-limiting example of an alarm GUI displayed by selecting an alarm button in the patient activity GUI of FIG. 6A;

FIG. 6D illustrates a non-limiting example of a threshold selection GUI displayed via the patient activity GUI of FIG. 6A;

FIG. 6E illustrates a non-limiting example of a condition selection GUI displayed via the patient activity GUI of FIG. 6A;

FIG. 6F illustrates a non-limiting example of a histogram GUI displayed via the patient activity GUI of FIG. 6A;

FIG. 6G illustrates a non-limiting example of a patient activity graphical user interface (GUI);

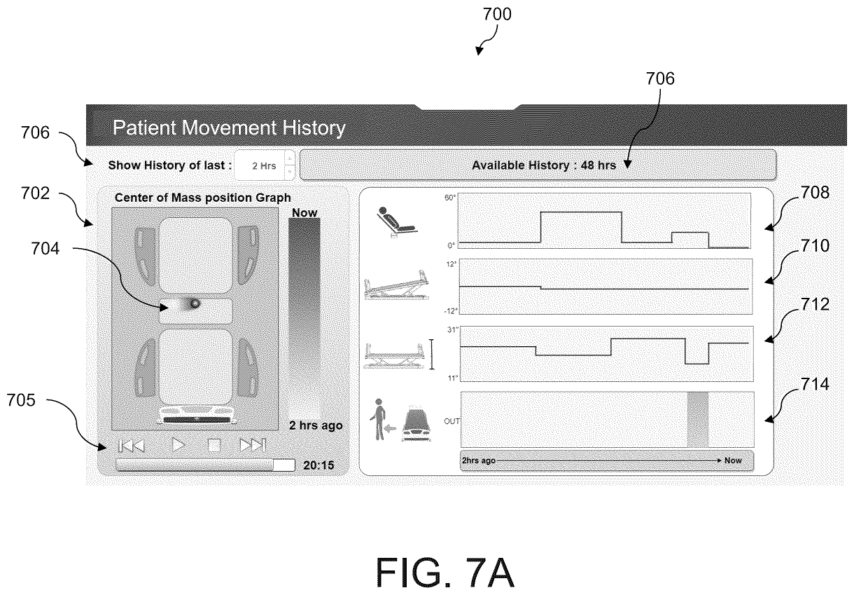

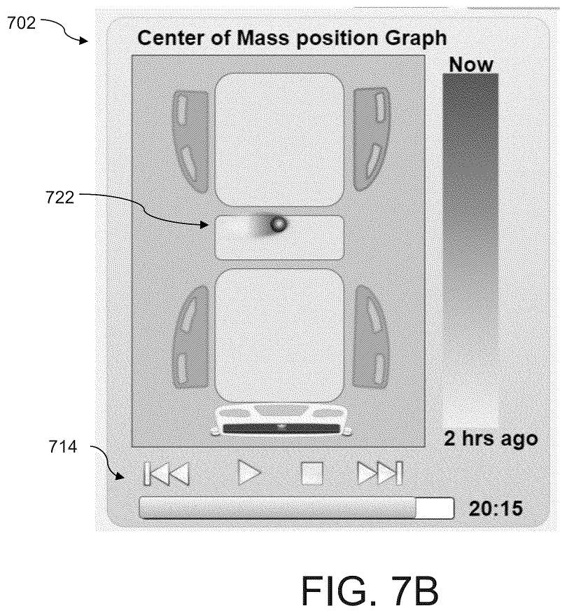

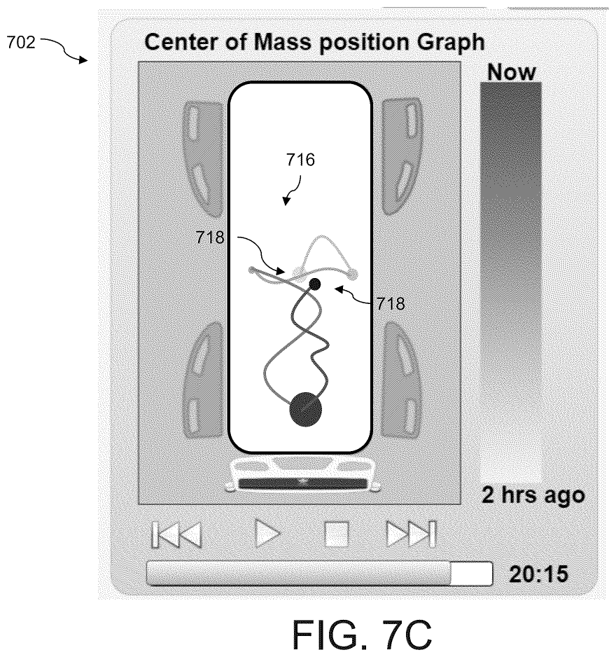

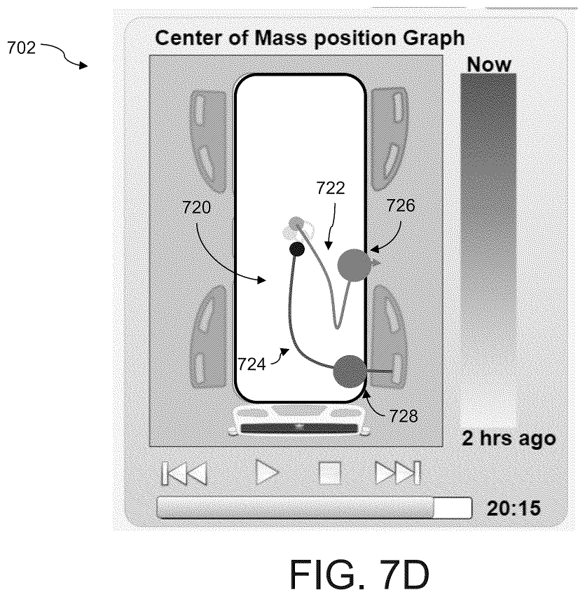

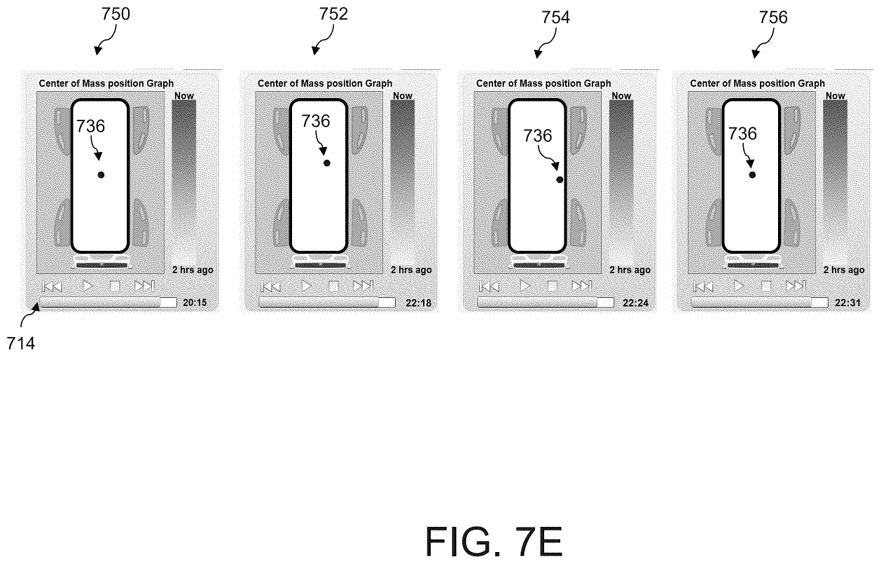

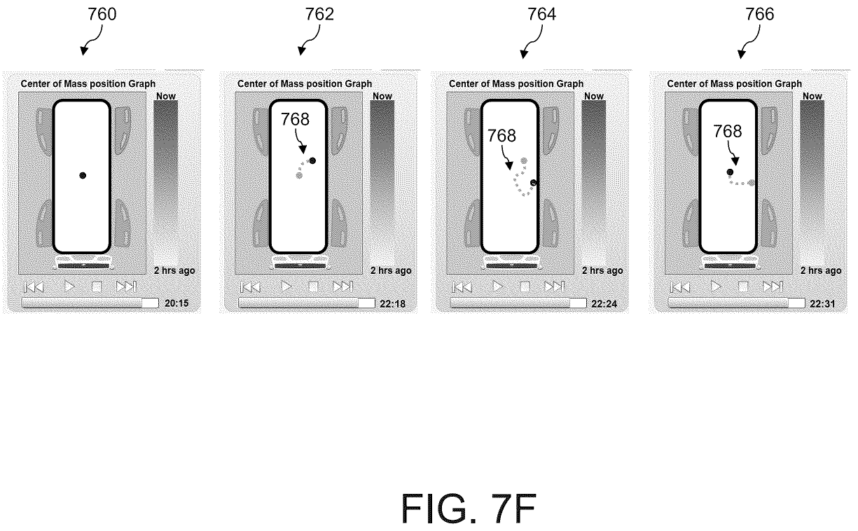

FIG. 7A illustrates a non-limiting example of patient movement history GUI having a center of mass position graph and timelines of an angle of backrest, a position of the bed, a height of the bed and exits and entrances from the bed;

FIG. 7B illustrates a non-limiting example of a center of mass position graph which may be displayed as part of the patient movement history GUI of FIG. 7A;

FIG. 7C illustrates a non-limiting example of historical positions of the patient center's mass displayed in the center of mass position graph;

FIG. 7D illustrates another non-limiting example of historical positions of the patient center's mass displayed in the center of mass position graph;

FIG. 7E illustrates non-limiting examples of consecutive positions of the patient center's mass in time viewed using the navigation controls of the patient movement history GUI;

FIG. 7F illustrates additional non-limiting examples of consecutive positions of the patient center's mass in time viewed using the navigation controls of the patient movement history GUI;

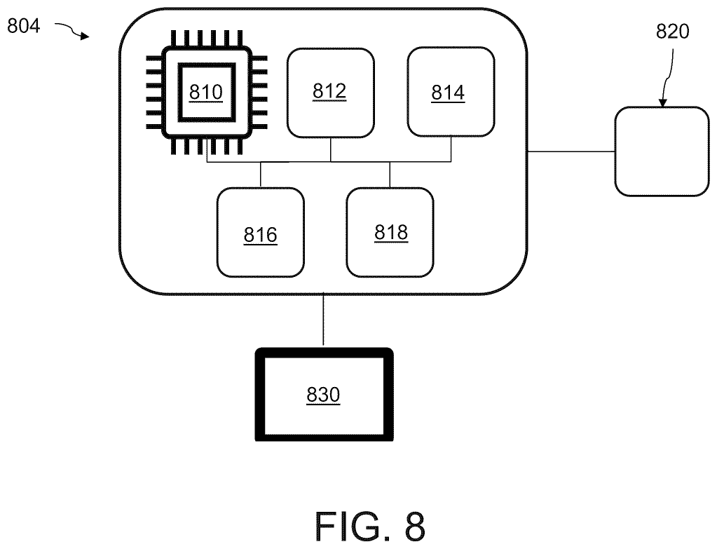

FIG. 8 illustrates a computing device connected to one or more load cells and to a display device in accordance with one or more non-limiting implementations of the present technology;

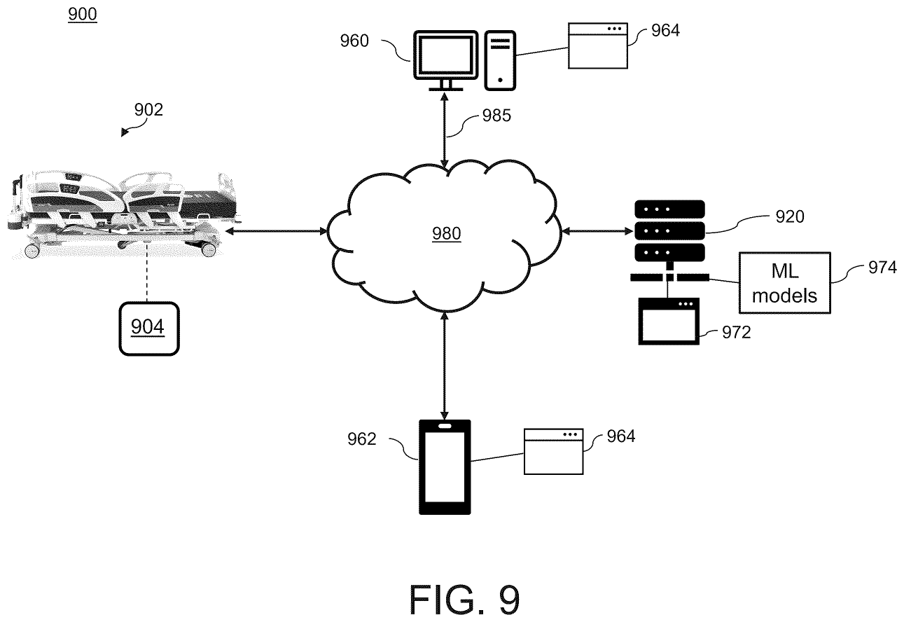

FIG. 9 illustrates an environment and system in accordance with one or more non-limiting implementations of the present technology;

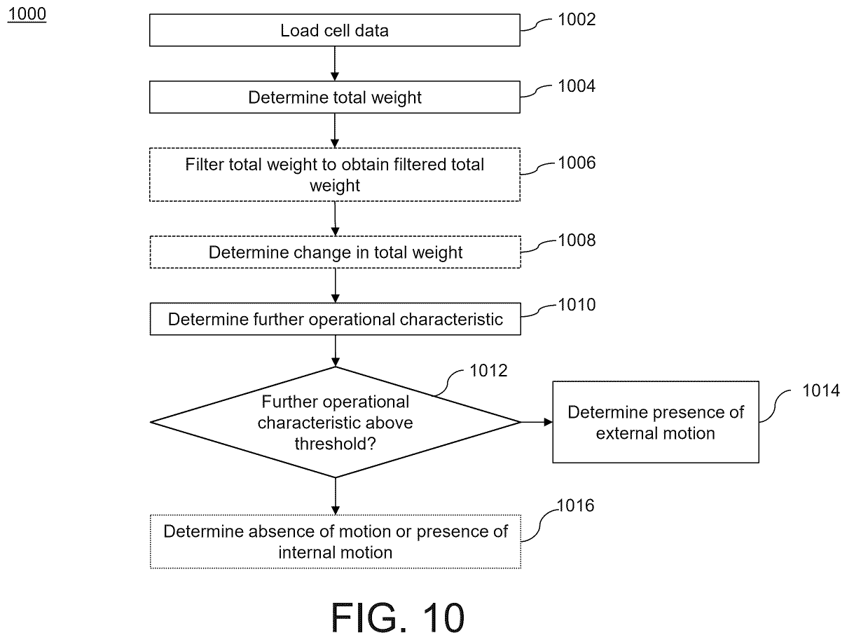

FIG. 10 illustrates a flowchart of a method of determining a presence of external motion or presence of internal motion/absence of motion in accordance with one or more non-limiting implementations of the present technology;

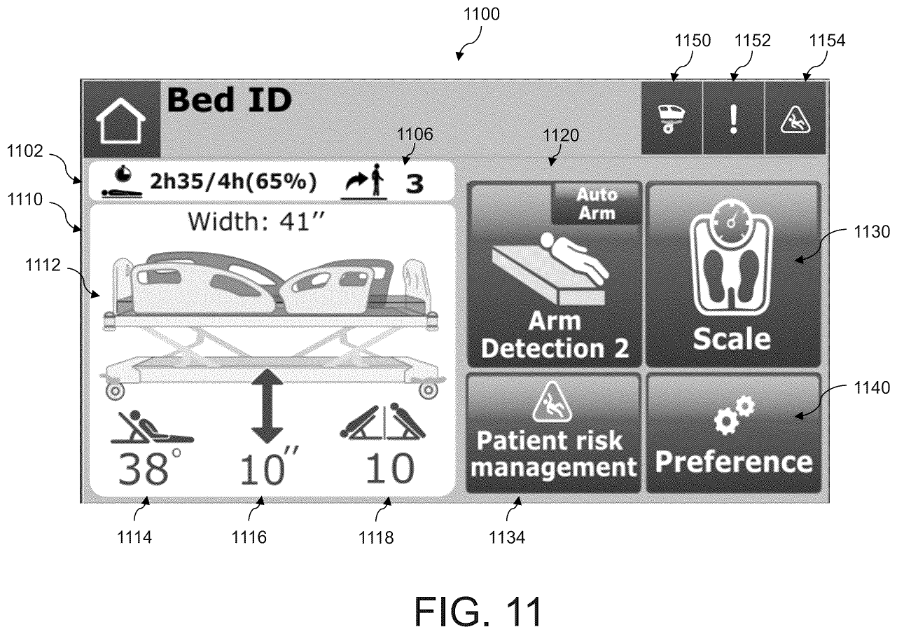

FIG. 11 illustrates a non-limiting example of a home GUI;

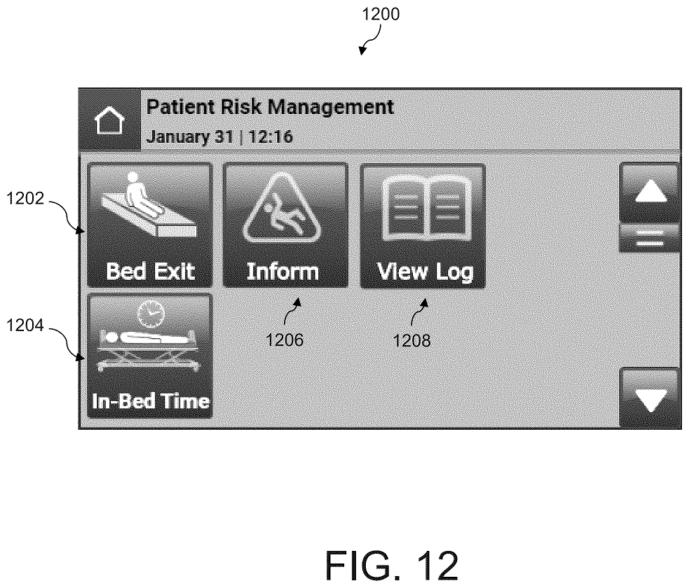

FIG. 12 illustrates a non-limiting example of a patient risk management GUI displayed by selecting a patient risk management button on the home GUI of FIG. 11;

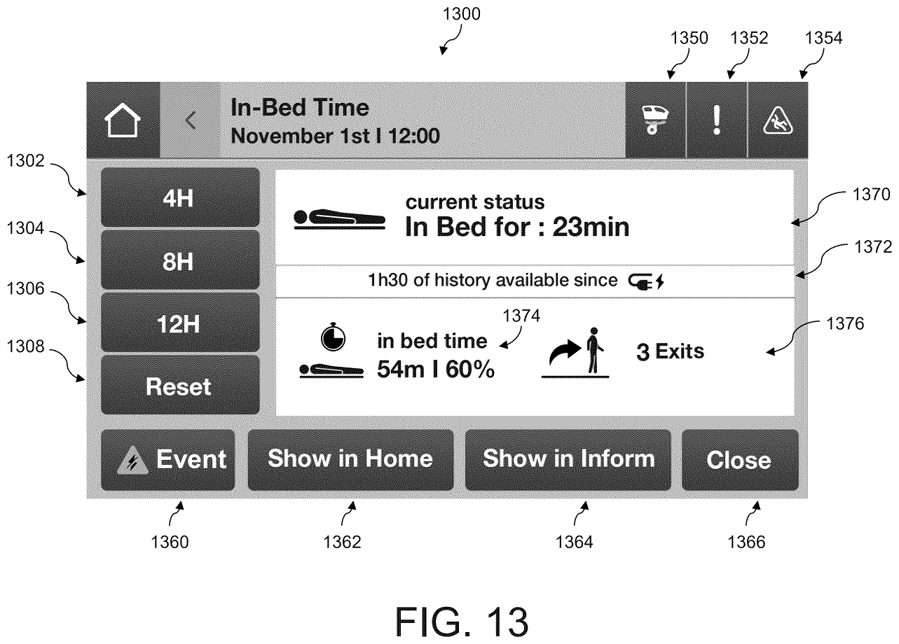

FIG. 13 illustrates a non-limiting example of an in-bed time GUI displayed by selecting an in-bed time button on the home GUI of FIG. 11;

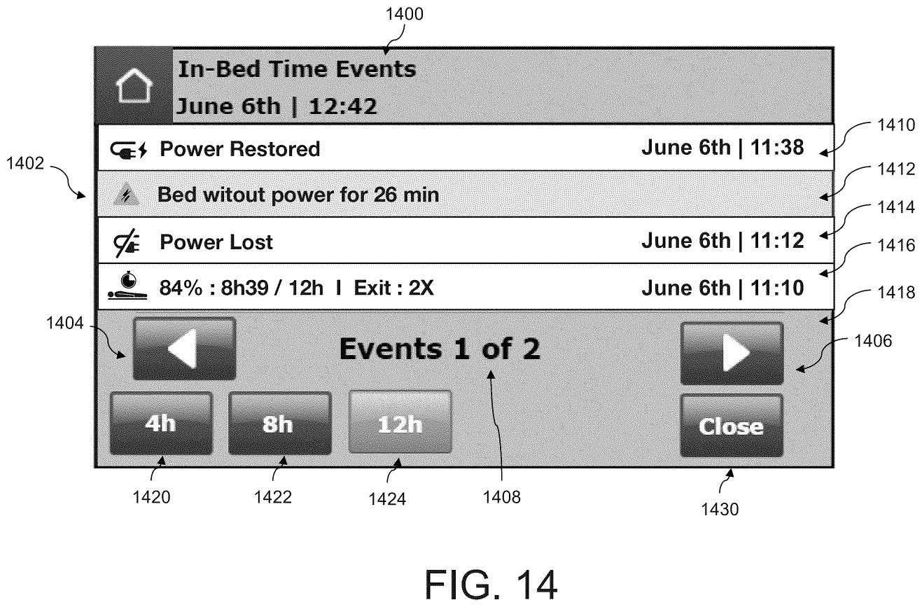

FIG. 14 illustrates a non-limiting example of an in-bed time events GUI displayed by selecting an in-bed time events button on the in-bed time GUI of FIG. 13;

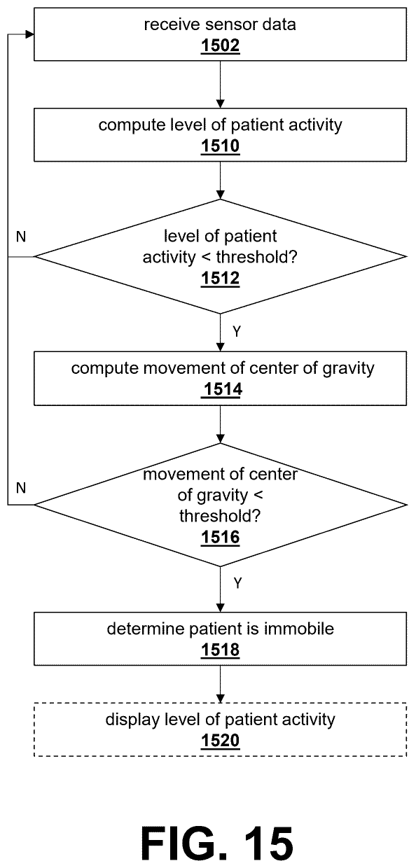

FIG. 15 illustrates a flow chart of a method for determining immobility of a patient in accordance with one or more non-limiting embodiments of the present technology;

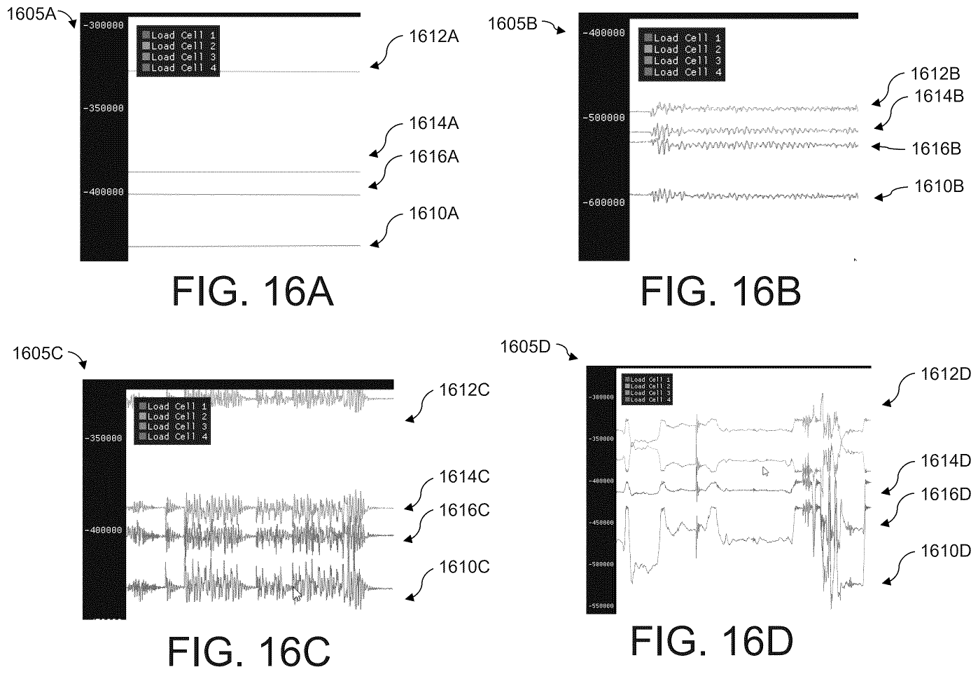

FIG. 16A illustrates a non-limiting example of a graph with force measurements of four load cells which correspond to levels of patient activity between 0 to 1 on a scale of 0 to 5 in accordance with one or more non-limiting embodiments of the present technology;

FIG. 16B illustrates a non-limiting example of a graph with force measurements of four load cells which corresponds to levels of patient activity between 1 to 2 on a scale of 0 to 5 in accordance with one or more non-limiting embodiments of the present technology;

FIG. 16C illustrates a non-limiting example of a graph with force measurements of four load cells which corresponds to levels of patient activity between 2 and 3 on a scale of 0 to 5 in accordance with one or more non-limiting embodiments of the present technology;

FIG. 16D illustrates a non-limiting example of a graph with force measurements of four load cells which corresponds to levels of patient activity between 3 to 5 on a scale of 0 to 5 in accordance with one or more non-limiting embodiments of the present technology;

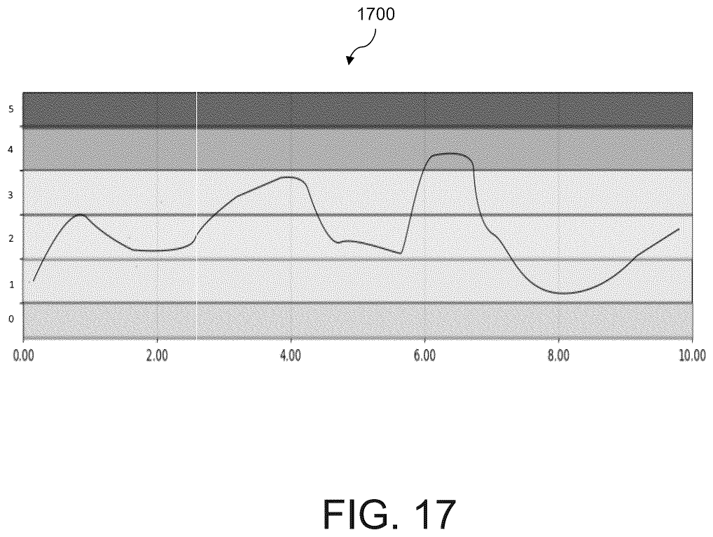

FIG. 17 illustrates an example of a patient activity graph in accordance with one or more non-limiting embodiments of the present technology;

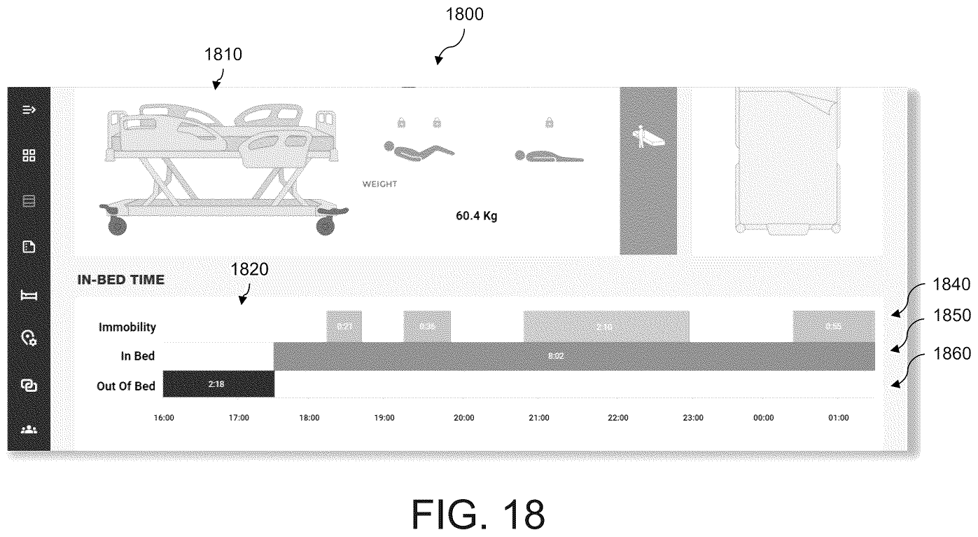

FIG. 18 illustrates a non-limiting example of a patient movement GUI in accordance with one or more non-limiting embodiments of the present technology;

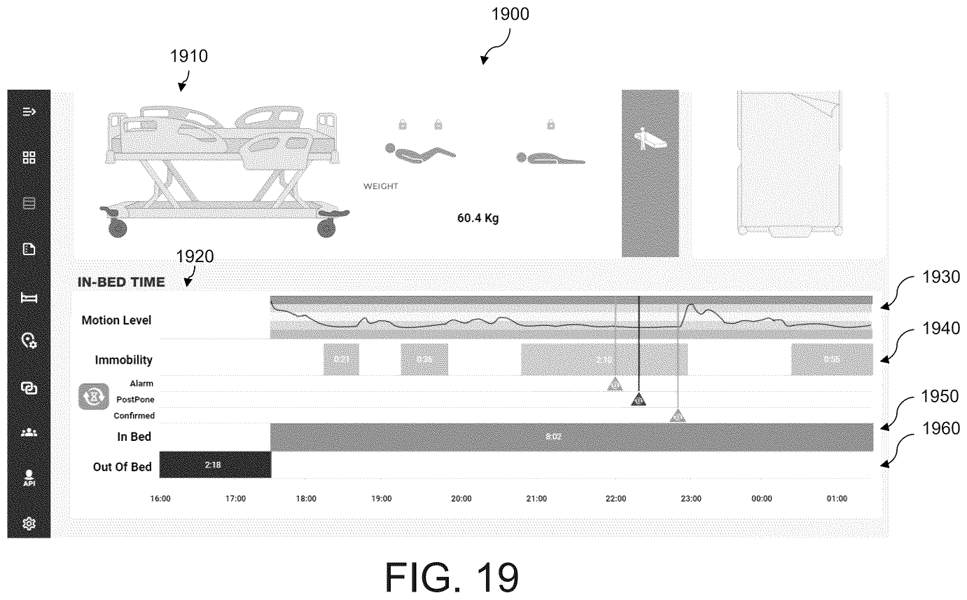

FIG. 19 illustrates another non-limiting example of a patient movement GUI in accordance with one or more non-limiting embodiments of the present technology;

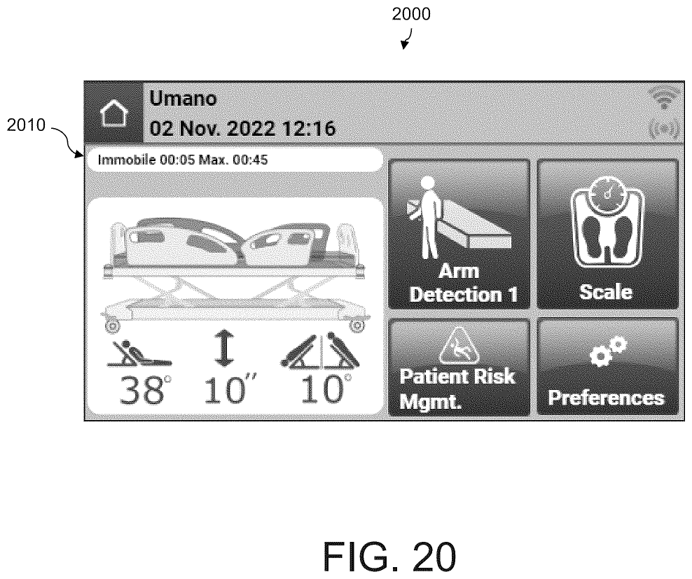

FIG. 20 illustrates an example of a home interface GUI with immobility in accordance with one or more non-limiting embodiments of the present technology;

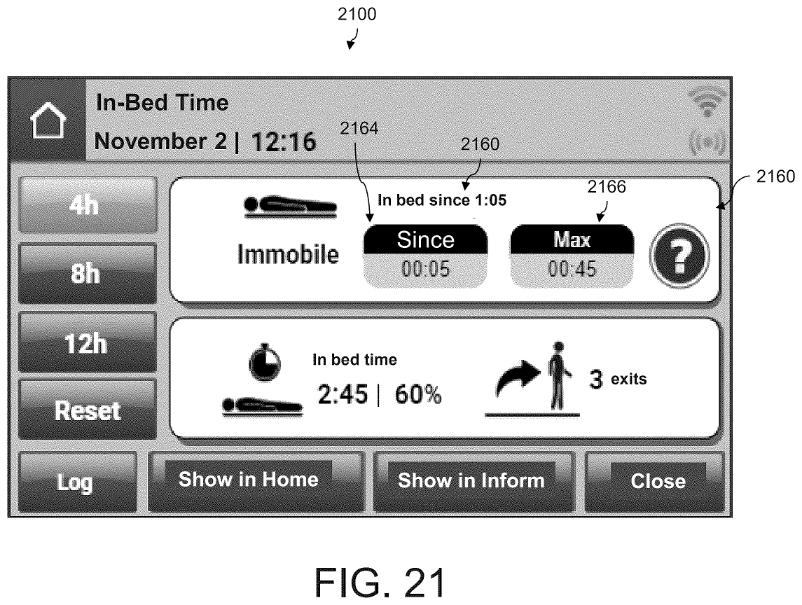

FIG. 21 illustrates an embodiment of an in-bed time GUI with immobility; and

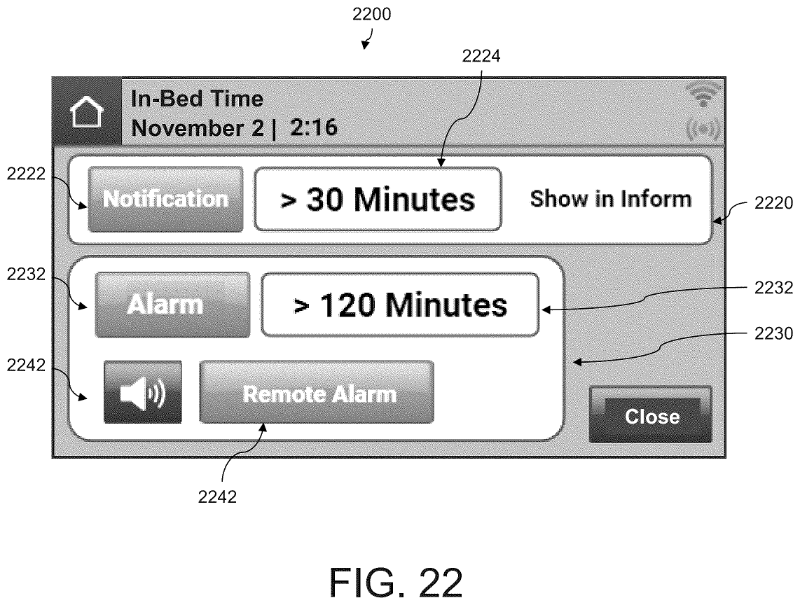

FIG. 22 illustrates an embodiment of an immobility management GUI.

### DETAILED DESCRIPTION

Referring to FIG. 1, there is shown a patient support apparatus in the form of hospital bed 100, in accordance with one or more implementations of the present technology. While in FIG. 1, the patient support apparatus is depicted as the hospital bed 100, the patient support apparatus may be implemented as an intensive care unit (ICU) bed, a bariatric bed, a reclining chair, a stretcher, or any other form of support apparatus configured to support at least a portion of a body of a patient and configured to receive and use force sensing devices, without departing from the scope of the present technology.

The bed 100 has a head end 102, an opposite foot end 104 and spaced-apart left 105 and right 107 sides extending between the head end 102 and the foot end 104.

Some of the structural components of the bed 100 will be designated hereinafter as “right”, “left”, “head” and “foot” from the reference point of an individual lying on his/her back on the support surface of the mattress provided on the bed 100 with his/her head oriented toward the head end 102 of the bed 100 and his/her feet oriented toward the foot end 104 of the bed 100.

The bed 100 includes a base 106, a patient support assembly 108 and an elevation system 110 operatively coupling the patient support assembly 108 to the base 106. In the illustrated implementation, the patient support assembly 108 includes a frame 109 and a patient support surface 111 supported by the frame 109. In the illustrated implementation, the patient support surface 111 includes an upper body surface or backrest 113, a lower body surface or lower body support panel 115 and one or more core body surfaces or core support panels 117, 119 located between the backrest 113 and the lower body support panel 115 for supporting the seat and/or thighs of the patient. In the illustrated implementation, each one of the backrest 113, the lower body support panel 115 and the core support panels 117, 119 can be angled relative to the other panels. Alternatively, the patient support surface 111 could comprise a single rigid panel extending between the head end 102 and the foot end 104 of the bed 100 instead of multiple pivotable panels.

The bed 100 further includes a patient support barrier system 120 generally disposed around the patient support assembly 108. The barrier system 120 includes a plurality of barriers which extend generally vertically around the patient support assembly 108. In the illustrated implementation, the plurality of barriers includes a headboard 122 located at the head end 102 and a footboard 124 disposed generally parallel to the headboard 122 and located at the foot end 104 of the bed 100. The plurality of barriers further includes spaced-apart left and right head siderails 126, 128 which are located adjacent the headboard 122 and spaced-apart left and right foot siderails 130, 132 which are respectively located between the left and right head siderails 126, 128 and the foot end 104 of the bed 100. Each one of the plurality of barriers is moveable between an extended or raised position for preventing the patient lying on the bed 100 from moving laterally out of the bed 100, and a retracted or lowered position for allowing the patient to move or be moved laterally out of the bed 100.

The hospital bed 100 includes a control unit 180 (schematically shown), also referred to as controller 180. The controller 180 is operatively connected to different systems and sub-systems of the bed including inter alia the elevation system 110, a plurality of pivoting systems (not numbered) and a plurality of sensors (not shown) and configured to receive and transmit signals therewith. The controller 180 may be operatively connected to the components of the hospital bed 100 via one or more circuitries (not shown). In one or more embodiments, different systems and sub-systems of the hospital bed 100 are configured to transmit data via a data bus (not shown) operatively connected to the controller 180. The controller 180 is used to control various functions of the hospital bed 100. In one implementation, the controller 180 is mounted on the patient support assembly, for example below one of the panels of the patient support surface. It will be appreciated that the controller 180 may be provided at different locations, may be integrated into the bed 100 or may be a separate device operatively connected to at least one component of the bed 100.

The controller 180 comprises one or more processors, one or more memories, one or more input/output interfaces and communication interfaces (not shown). It will be appreciated that the controller 180 is an implementation of a computing device. A non-limiting example of how the controller 180 is implemented will be provided hereinafter with reference to FIG. 8.

The hospital bed 100 may further include a control interface (not shown) operatively connected to the controller 180 and configured for receiving user inputs for controlling features of the bed 100 and outputting information relating to the features of the bed 100 and/or the patient. The control interface could be integrated into the footboard 124, into the headboard 122 or into one or more of the siderails 126, 128, 130, 132. Alternatively, the control interface could be provided as a separate unit located near the bed 100 or even at a location remote from the bed 100.

The bed 100 may further comprise a plurality of wheels 150 and a brake system (not shown) operatively coupled to the wheels 150. The brake system is configured to be able to immobilize the bed 100 and prevent rolling of the wheels 150.

The bed 100 further comprises a weight measurement system (not shown) configured for measuring the weight of the patient lying on the bed 100. Specifically, the weight measurement system (not shown) is provided in the base 106 of the bed 100. In one or more implementations, the weight measurement system is operatively connected to the controller 180.

As shown in FIG. 2, in the illustrated implementation, the base 106 is generally rectangular and comprises a fixed frame 200 and a suspended frame 202 movably connected to the fixed frame 200. The suspended frame 202 comprises parallel left and right longitudinal members 204, 206 and parallel head and foot transversal members 208, 210 which extend between and connect the left and right longitudinal members 204, 206 at the head and foot ends 102, 104 of the bed 100, respectively. More specifically, the left longitudinal member 204 is connected to the head transversal member 208 at a left head corner 212 of the suspended frame 202 and to the foot transversal member 210 at a left foot corner 214 of the suspended frame 202. Similarly, the right longitudinal member 206 is connected to the head transversal member 208 at a right head corner 216 of the suspended frame 202 and to the foot transversal member 210 at a right foot corner 218 of the suspended frame 202.

In the illustrated implementation, each one of the left and right longitudinal members 204, 206 and each one of the head and foot transversal members 208, 210 is hollow and has a generally rectangular cross-section. It will be appreciated that this configuration provides the suspended frame 202 with relatively good resistance to bending and torsion while allowing the suspended frame 202 to have a relatively low weight.

The suspended frame 202 further includes corner braces 220 connecting adjacent transversal and longitudinal members. The corner braces 220 brace the suspended frame 202 by maintaining the transversal members 208, 210 perpendicular to the longitudinal members 204, 206, and are also adapted to be pivotably connected to the lower ends of the pivoting links that support the patient support assembly 108. The suspended frame 202 further comprises head and foot actuator brackets 222, 224 extending downwardly from the head and foot transversal members, respectively. The head actuator bracket 222 and the foot actuator bracket 224 are adapted to be pivotably connected to the elevation assembly 110.

The fixed frame 200 comprises parallel left and right longitudinal members 250, 252 and parallel head and foot transversal members 254, 256 which extend between and connect the left and right longitudinal members 250, 252 at the head and foot ends 102, 104 of the bed 100, respectively.

The head and foot transversal members 254, 256 of the fixed frame 200 have a U-shaped cross-section and are spaced from each other by a distance D₁, and the head and foot transversal members 208, 210 of the suspended frame 202 are spaced from each other by a distance D₂ which is smaller than the distance D₁. This configuration allows the suspended frame 202 to fit within the fixed frame 200. Specifically, the distances D₁ and D₂ are selected such that the head transversal member 208 of the suspended frame 202 is adjacent the head transversal member 254 of the fixed frame 200, and that the foot transversal member 210 of the suspended frame 202 is adjacent the foot transversal member 256 of the fixed frame 200.

The base 106 further comprises a plurality of load cells which are adapted to connect the suspended frame 202 to the fixed frame 200 and configured to provide an indication of the weight on the bed 100. In the illustrated implementation, the base 106 includes four load cells 260 (two of which can be seen in FIG. 2), each disposed near one of the corners 212, 214, 216, 218 of the suspended frame 202. It is contemplated that the bed 100 may have a different number of load cells 260, for example only a single load cell 260, or that the one or more load cells 260 may be positioned at different locations on the bed 100. Multiple load cells 260 may be desired for other purposes, such as determining a position of the patient in the bed 100.

The load cells 260 may be of any suitable design, such as co-planar beam load cell model 380 manufactured by Vishay Precision Group Inc. (Malvern, U.S.A.), or type PB planar beam load cell manufactured by Flintec Inc. (Hudson, U.S.A.). Additional details of the load cells 260 and the bed 100 are disclosed in U.S. Pat. No. 10,117,798 by the same Applicant, which is incorporated by reference herein in its entirety.

The one or more load cells 260 are operatively connected to the controller 180.

In some implementations, the one or more load cells 260 may be used to acquire, detect and/or enable the determination of various types of measurements, including weight measurement of objects or individuals on the bed 100 or in proximity thereto, force measurements to quantify applied forces such as tension and compression, pressure data, center of mass determination, and load distribution analysis across surfaces or among multiple load cells. Further, the one or more load cells 260 may be used for counting and identifying patients and for inventory management.

In some implementations, in addition to the vertical force components (i.e., y-axis), force components in other directions may be used in the context of the present technology to determine the level of activity.

A mattress (not shown) is typically provided and removably attached to the hospital bed 100. It is contemplated that different sized mattresses may be provided depending on the width configuration of the hospital bed 100. For example, the hospital bed 100 may accommodate a 35 inch (890 mm) wide mattress. An adjustable width bed, for example a bed suitable for bariatric patients, may accommodate a 35-inch-wide mattress in the narrow configuration and a 45 inch (1140 mm) wide mattress in the wide configuration.

In the context of the present technology, the hospital bed 100 equipped with the one or more load cells 260 is used to determine, track and predict the activity level of a patient occupying the hospital bed 100. For example, a low level of activity or movement may indicate a risk for bedsores or other conditions. A high level of movement may indicate an aggressive patient or a reaction to a medication. A change in movement level during sleep may indicate a patient in need of assistance. A loss of activity by a patient in palliative care may indicate that a patient has passed away. Any level of movement can help distinguish patient presence in the bed from the weight of accessories or other inanimate objects. Observed patient activity levels can be used to predict future patient activity levels, for example predicting aggressive behavior, or predicting that the patient's future activity levels will be higher or lower than the activity level recommended by a medical professional (e.g., a physician). Other purposes for monitoring patient activity levels will be apparent to persons of ordinary skill in the art.

In one or more implementations, data from the load cells 260 enables determining weight shift detection by analyzing data from load cells to identify patient movements or changes in position of the patient.

In one or more implementations, data from the load cells 260 enables determining bed entry and exit times by monitoring the load cells to detect when a patient gets into or out of the bed.

In one or more implementations, data from the load cells 260 enables performing pressure distribution analysis by evaluating the pressure data across the bed surface to assess the patient's posture and potential risk areas for pressure ulcers.

In one or more implementations, data from the load cells 260 enables determining movement patterns by analyzing the frequency and nature of patient turning or repositioning, indicating the ability of the patient to move independently.

In one or more implementations, data from the load cells 260 enables determining respiration rate by detecting subtle movements associated with breathing through load cell data, providing an indirect indicator of patient mobility and health status.

In one or more implementations, data from the load cells 260 enables determining restlessness or agitation by identifying frequent or irregular movements.

In one or more implementations, data from the load cells 260 enables determining sleep quality by monitoring and analyzing patient movements during sleep.

In one or more implementations, patient activity levels may be expressed as mobility levels according to the Braden scale standard designed to assess the mobility of a patient supported by a patient support apparatus.

The Braden scale is a clinical tool known in the art for assessing the risks of patients developing pressure ulcers in bedridden patients. The Braden scale consists of six subscales, one of which is specifically focused on patient mobility. Each subscale is rated from 1 to 4, and an overall score is then calculated to evaluate the overall risk of pressure ulcers of a patient.

It will be appreciated that the determination of events and degrees of activity using data from the load cells may depend on whether sufficient power is available and/or a continuous source of power is available. For instance, the determination of events and degrees of activity may be performed differently whether the patient support apparatus is connected to an external power source such as an electrical power grid of a facility (e.g., hospital) or if it is powered by a battery source (e.g., battery). In some implementations, such as when the patient support apparatus is powered by a battery, the determination of events and degrees of activity using data from the load cells 260 may be performed at lower frequencies (i.e., at fewer intervals). In such implementations, an indication that the determination of the events or level of activity may not be reliable or that it has been performed at lower frequencies may be displayed or otherwise transmitted to a user viewing the information determined using the data from the load cells 260.

In one or more other implementations, the determination of events and degrees of activity using data from the load cells 260 may not be performed to save resources to power other functionalities of the bed 100. In such implementations, an indication that the determination of the events or level of activity is not available may be displayed or otherwise transmitted to a user viewing the information determined using the data from the load cells 260.

Referring to FIG. 3, a flowchart of a method 300 of determining a level of patient activity within the bed is shown, according to an implementation.

The method 300 may be performed by software or hardware integrated in the bed, by a remote software application receiving load cell data from the bed, or by a system including the hospital bed and a remote software application running on an electronic device that can be connected to the bed via a communications network, for example.

In one or more implementations, the method 300 may be stored in the form of computer-readable instructions within one or more non-transitory storage mediums. The computer-readable instructions, upon being loaded and executed by one or more processors, cause the execution of the method 300.

In one or more implementations, the method 300 is executed by one or more processors operatively connected to the load cells 260. In one or more other implementations, the method 300 may be executed by a plurality of processors. Non-limiting examples of computing devices and environments and systems for executing the method 300 are provided hereinafter with reference to FIG. 8 and FIG. 9.

At processing step 302, sensor data is collected from the load cells 260. The sensor data may include a plurality of readings taken by each of the load cells 260 at regular intervals. It is contemplated that the sensor data may take other forms, such as a total weight detected by the load cells 260 at each time interval, or indications of changes in weight relative to a previous time interval. The sensor data is transmitted from the load cells 260 and received by one or more processors (e.g., one or more processors of the controller 180 of the bed 100 or of another computing device).

At processing step 304, a total weight detected by the plurality of load cells 260 is determined. The total weight is determined for each time interval at which sensor readings have been acquired by the load cells 260. This determination may be performed concurrently with receiving the sensor data at processing step 302, for example if the total weight was received from the load cells 260 at processing step 302, or if there is only a single load cell 260. In some implementations, the total weight may be determined by a processing component of the plurality of load cells 260, the controller 180 of the bed or another computing device.

At processing step 306, a change in the total weight is determined. The change in the total weight may be an absolute change in the total weight detected by the plurality of load cells 260 between consecutive weight measurements. The change in the total weight may be a rate of change or a relative change (e.g., a percentage change) in the total weight detected by the plurality of load cells 260 between consecutive weight measurements. The change in total weight may be determined for a plurality of time intervals for which sensor data is received. A duration of the change in weight may also be determined.

In some implementations, the sensor data may be filtered using techniques such as noise reduction (e.g., a low pass filter), signal smoothing, baseline drift corrections, artifact rejection and the like.

At processing step 308, an operational characteristic is determined, based on at least one of the magnitudes of the changes in total weight or the time duration of the changes in total weight.

The operational characteristic may be any suitable function of at least one of the magnitudes of the changes in total weight or the time duration of the changes in total weight, such as a variance or a range of the weight over a period of time. The operational characteristic may also include or be based on a rate of change of weight, a standard deviation, a weight change frequency, a cumulative weight change, and a moving average.

Non-limiting examples of operational characteristics are described herein below in further detail.

At processing step 310, a level of activity of the patient is determined, based on the operational characteristic. Example methods of determining the level of activity of the patient will be described below in further detail.

The one or more processors determines, based on the operational characteristics, the level of activity.

In one or more implementations, the level of activity is determined by a trained machine learning (ML) model, as explained hereinafter. The trained ML model may receive as an input one or more of: the change in total weight and the operational characteristics, and may determine a level of activity of the patient in the time interval. In some implementations, the trained ML model (or another trained ML model) may predict a future level of activity of the given patient based on past and current activity of the given patient recorded via the load cells.

At processing step 312, the level of activity of the patient is optionally displayed on a display device to a user, such as a hospital employee. Example methods of displaying the level of activity of the patient will be described below in further detail.

In one or more implementations, one or more processors transmit signals to cause display of the level of activity of the patient, optionally with the output of any one of processing steps 302 to 310.

In one or more other implementations, the one or more processors may transmit an indication of the level of activity to another type of input/output device, such as an audio output device (e.g., speaker), a tactile output device, a printer, which may cause the input/output device to communicate the level of activity to a user in vicinity (e.g., medical personnel).

In one or more implementations, processing step 309 may be executed to determine presence of the patient in the patient support apparatus and time spent in the patient support apparatus by the patient based on the changes in the total weight detected over the given period of time. It should be understood that in some implementations, the presence of the patient in the patient support apparatus and time spent by the patient in the patient support apparatus may be determined with less data points over the given period of time than required for determining the level of activity at processing step 310. Optionally, at processing step 313, the one or more processors may transmit a signal to cause display of an indication of the presence of the patient in the patient support apparatus and the time spent in the patient support apparatus. Processing steps 309 and 313 may be executed at any time after processing step 306.

The presence of the patient in the patient support apparatus may be used to determine time spent by the patient in the patient support apparatus.

In one or more implementations, the method 300 for determining the level of activity may be executed for a plurality of patients in respective patient support apparatuses. In such implementations, the level of activity of each of the plurality of patients could be displayed to a client device (e.g., nurse station computer, client device or other type of computing device) together with the past level of activity of the patient. Thus, this may enable a medical professional to prioritize provision of care depending on the changes in level of activity,

In one or more implementations, the following example algorithm (“Algorithm 1”) can be used for determining the operational characteristic at processing step 308.

```
Loads is a 4xn array, n being the number of ms since
start of acquisition
  For load in loads :
    Load = smoothed(load)
  Weight_per_timestep = sum of all loads for each
timestep
  Weightvar = moving_variance(weight_per_ms,
  neighbors = 1000)
    or
  Minmax = moving_minmax(weight_per_ms, neighbors =
1000)
  Activity = log10(Weightvar) or log10(Minmax)
```

In this example, a sensor reading is received from each of the four load cells 260 every millisecond in the form an array of size 4×n. It should be appreciated that sensor data can be generated at any suitable frequency within the capability of the load cells 260, and that any number of load cells 260 may be used.

In this example, the received sensor data is optionally smoothed, using any suitable smoothing algorithm, to reduce the impact of outlier points that might be due to noise or other errors. Then, for each timestep representing one set of sensor readings (each 1 ms in this example), the weights measured from each load cell sensor in that timestep are summed, to determine the total weight measured at that timestep. Algorithm 1 then determines one of a moving variance and a moving minimum and maximum of the weight per timestep for a window or interval of time steps (e.g., neighbors=1000 time steps in the above example).

The moving variance measures how much the weight readings vary over time within the time interval, and the moving min-max identifies the lowest and highest weight readings within a time interval.

Then, Algorithm 1 determines the activity level by calculating a logarithm (base 10) of the variance or a logarithm of the min-max. It should be understood that the logarithm step is used to scale data down to a manageable range and may be optional, and a different scaling may optionally be used. This result is then output as the activity level of the patient.

In one or more implementations, the following example algorithm (“Algorithm 2”) can be used for determining the variance of the total weight.

```
neighbors of type int
data of type array
For i in length(data):
  Interval = data(I − neighbors/2: i +
neighbors/2)
  Weightvar(i) = variance(interval)
Return weightvar
```

In this example, an array of data is received, representing the total weight for each timestep i, collected over a period of time by the load cell sensors. Then, at each timestep i, an interval is taken around the data, for example an interval of 1000 timesteps (i.e., neighbors). The variance of the data over the time interval is then determined and stored as an element in a weight variance variable. Algorithm 2 then outputs the variable comprising the weight variances determined for the intervals.

In one or more implementations, the following example algorithm (“Algorithm 3”) can be used for determining the minimum-maximum (min-max) of the total weight.

```
neighbors of type int
data of type array
For i in length(data):
  Interval = data(i − neighbors/2: i +
neighbors/2)
  Minmax(i) = (max(interval) − min(interval))\hat{ }2
Return Minmax
```

In this example, an array of data is received, representing the total weight for each timestep i, collected over a period of time. Then, at each timestep i, an interval is taken around the data, for example an interval of 1000 neighbors corresponding to 1000 timesteps. The maximum value and minimum values of the total weight over the interval in the array are determined, and their difference is computed. The difference between the minimum weight and the maximum weight is the range of the total weight over the time interval. The square of the range is then output as the “min-max” for each interval. Alternatively, the range itself may be output and used for the purposes described herein.

The following example algorithm (“Algorithm 4”) can be used for detecting patient activity events based on weight detection.

```
  Thresholds = [thresh1, thresh2, thresh3 ...] in decreasing
order
  For i in length(thresholds):
    Starts, ends = where(activity crosses
  thresholds(i))
    For j in length starts:
      If a center exists already between starts(j)
      and ends(j):
    Skip this interval : it was already measured at a
    higher threshold
      Centers.append(starts(j) + ends(j))//2)
      Movement = centermass(ends(j)) −
      centermass(starts(j))
      Events.append({thresholds(i), amplitude,
    movement})
  Return events
```

Algorithm 4 may receive as an input the activity level determined by executing Algorithm 1.

Algorithm 4 receives as an input an array including one or more thresholds, which may be predetermined levels of patient activity. Algorithm 4 executes a for loop iterating over each threshold in the threshold array. The Algorithm 4 identifies time periods during which each of the threshold has been crossed. A start time and end time are determined, representing the beginning and end of a time interval when the threshold is crossed. It should be understood that the event of interest may be a detection of activity higher than a threshold, for example to identify seizures or reactions to medication, or the event of interest may be a detection of activity lower than a threshold, for example to determine when a previously agitated patient has calmed down.

In Algorithm 4, once the algorithm determines the start time and end time of the event, the Algorithm 4 determines the center time of the event, which is the average of the start time and the end time. The center time is then recorded, along with other relevant information about the event, such as one or more of the amplitude of the event, the duration of the event, and the movement of the patient's center of mass during the event. However, if there already exists a center time within the interval of the detected event, Algorithm 4 may determine that this event has already been recorded based on a different threshold, and the event does not need to be recorded again. Thus, each event is recorded only with reference to the highest threshold that was crossed. By executing Algorithm 4, events corresponding to respective thresholds can be determined.

The following example algorithm (“Algorithm 5”) can be used for detecting longer-term patient activity.

```
Longterm activity (LA):
Inertia is a float of the order or 2e−5/ms −> 0.02/s
  For i in length(data):
    LA.append(LA(end)*(1-inertia) +
  data(i)*inertia)
  Return LA
```

According to Algorithm 5, a moving average of the activity level is determined by computing a weighted average of the previous average activity level and a current activity level. In this example, the value of the “inertia” variable is small (2*10−5 to 0.02), and as a result a small or short-duration change in activity level will only have a small effect on the activity level. It will be appreciated that Algorithm 5 doesn't require maintaining large amounts of previously received sensor data in memory, and the calculation can be performed quickly and efficiently without having to collect data over multiple timesteps.

Referring to FIGS. 4A to 4F, non-limiting example of graphs of patient activity level generated by executing the methods and algorithms disclosed herein are shown for various scenarios illustrative of typical patient activity levels, where the x-axis represents time in seconds(s) and the y-axis represents activity level (no units). In the graphs 400A, 400B, 400C, 400D, 400E and 400F, the current activity level is represented by the upper curves 402A, 402B, 402C, 402D, 402E, and 402F, with curves 404A, 404B, 404C, 404D, 404E, and 404F representing the smoothed activity level, and the long-term activity level is represented by the lower curves 406A, 406B, 406C, 406D, 406E, and 406F which may be grayscale coded or color coded based on activity level or have high-activity periods indicated by arrows or other types of graphical indicators.

Referring to FIG. 4A, an example graph 400A of activity level over time is shown, in which the hospital bed is not occupied by a patient. The activity peak at approximately 300 s corresponds to the head portion of the hospital bed being raised to an angle of 30 degrees. This activity peak does not appear on curve 404A or otherwise marked on the graph 400A because the activity peak is due to a function of the bed and/or caused by an external motion.

Referring to FIG. 4B, an example graph 400B of activity level over time is shown, in which the hospital bed is occupied by a calm patient. The peaks at the beginning and end of the displayed time (at approximately 20 s and 340 s) marked by indicators 408B correspond to the patient entering and leaving the hospital bed.

Referring to FIG. 4C, an example graph 400C of activity level over time is shown, in which peaks of the curve 402C marked by indicators 408C correspond to a patient occupying the hospital bed kicking the bed at approximately ten-second intervals.

Referring to FIG. 4D, an example graph 400D of activity level over time is shown, in which a patient occupying the hospital bed simulates convulsions in which peaks are marked by indicators 408D.

Referring to FIG. 4E, an example graph 400E of activity level over time is shown, in which a patient occupying the hospital bed moves his hands occasionally, which is marked by indicators 408E.

Referring to FIG. 4F, an example graph 400F of activity level over time is shown, in which a person walks near the hospital bed.

Referring to FIG. 5, an example method 500 of processing weight sensor data of a patient support apparatus such as a hospital bed is described. The method 500 can be performed by hardware or software integrated into the hospital bed, or by a remote application.

In one or more implementations, the method 500 may be stored in the form of computer-readable instructions within one or more non-transitory storage mediums. The computer-readable instructions, upon being loaded and executed by the one or more processors, cause the execution of the method 500.

In one or more implementations, the method 500 is executed by one or more processors operatively connected to the load cells 260. In one or more other implementations, the method 500 may be executed by a plurality of processors. Non-limiting examples of computing devices and environments and systems for executing the method 500 are provided hereinafter with reference to FIG. 8 and FIG. 9.

At processing step 502, the one or more processors determine whether one or more new weight values are available from the load cells 260. The weight value may be the total weight value determined by summing the weights measured by the one or more individual load cells. Alternatively, the weight values may be the individual weights measured by the one or more individual load cells, which can then be summed to determine the total weight value.

If a weight value is received, the method 500 proceeds to processing step 504.

If no weight value is received, the method 500 remains at processing step 502.

It is contemplated that the method 500 may optionally proceed to processing step 504 only after a predetermined number of weight values have been received, or that the weight values may be buffered for multiple measurement periods and received in batches by the one or more processors executing the method 500. However, in this example the method 500 proceeds to processing step 504 after each time interval representing the periodicity of the measurement by the load cells, which may result in a more frequent determination of the activity level, which may also be more responsive to rapid changes in patient activity.

At processing step 504, the received weight values are added to a circular buffer by the one or more processors. The circular buffer permits the most recent weight values to be stored, while discarding older weight values to reduce data storage requirements. It should be understood that the circular buffer is capable of storing at least as many data points as are used by the method to determine the patient activity level.

In alternative implementations, all weight data may be stored without using a circular buffer.

At processing step 506, the circular buffer is scanned to retrieve the weight data corresponding to the time window to be used for the activity determination.

At processing step 508, the one or more processors determine if there are enough data points in the circular buffer to cover the time window. This step may be omitted if the method 500 has already been performed and it is known that there are enough data points in the circular buffer. If there are enough data points, the method 500 proceeds to processing step 510. If not, the method 500 returns to processing step 502 to collect additional data. It will be appreciated that to determine if there are enough data points as part of processing step 508, the number of data points in the circular buffer may be compared to a threshold, and in response to the number exceeding the threshold, the method 500 advances to processing step 510.

At processing step 510, the one or more processors compute a variance of the sampled data points. This may be done using any of the methods and algorithms described above. It is contemplated that the variance may be or include any suitable measure of a short-term change in the total weight, such as the min-max described above. In one example, the variance may be computed using equation (1):

$$Variance = \frac{{\sum }_{1}^{n} {Weight}_{n}^{2}}{n} = {Mean}^{2}$$

The mean may be calculated using equation (2):

$$Mean = \frac{{\sum }_{1}^{n} {Weight}_{n}}{n}$$

Additionally, the raw motion may optionally be determined using equation (3):

$$MotionRaw = {log}_{10} ( Variance - SystemVariance )$$

Where System Variance is the variance measured for the bed with no patient present, for example during a calibration phase.

At processing step 512, the motion level is optionally smoothed. In one or more implementations, the motion level is smoothed by using equation (4):

$$MotionSmoothed = α \cdot MotionRaw + ( 1 - α ) \cdot PreviousMotion$$

Where a is a predetermined weight, and PreviousMotion is a level of motion determined in an earlier iteration of processing step 510. This smoothing has the effect of reducing the prominence of sharp peaks, which increases the emphasis on longer-term activity events.

At processing step 514, the motion level is optionally normalized by patient weight. Any suitable normalization may be used, including a normalization of the raw motion or filtered motion to a desired numerical scale, such as a value between 0 to 4. In this example, 0 may represent a stationary patient, and 4 may represent the highest expected (or highest detectable) level of patient activity. In this manner, the activity level of the patient should be similar for different-sized patients who are similarly active or who are responding in a similar way to a similar stimulus. It has been observed that normalizing a logarithmic scale in units from 0 to 4 results in integer activity levels that correspond approximately to different qualitatively observable levels of patient activity. In one or more implementations, the motion level is represented using the Braden scale.

At processing step 516, the oldest data value is removed from the circular buffer, thereby enabling the circular buffer to accept a new data point.

At processing step 518, the one or more processors output the determined activity level for the patient. A history of activity levels over a period of time may be stored in a memory or type of storage medium, or displayed in a tabular or graphical format, for example. It is contemplated that processing step 516 and processing step 518 may be performed concurrently or in any order.

The method 500 then returns to processing step 502 to collect additional data.

In some implementations of the method 500, the one or more processors may obtain more frequent weight measurements, and may perform more frequent calculations, to obtain more frequent and more detailed information about the activity level of the patient. In other implementations, the one or more processors may obtain less frequent weight measurements, for example to optimize usage of computational resources or by using less expensive hardware or less computer memory.

Referring generally to FIGS. 6A to 6G, the patient activity information collected by any of the methods 300 and 500 described above can be displayed to a user such as a member of a hospital medical staff, for example in a printed report, on a screen disposed on patient support apparatus (e.g., the hospital bed 100), or via a remote application (app) that could optionally be accessed on desktop or remote devices. The hospital bed 100 may be connected to the remote app via a wireless communication network in the hospital. A non-limiting example of an environment and system 900 comprising a patient activity monitoring application 964 is described hereinafter with reference to FIG. 9.

Referring to FIG. 6A, there is shown a patient activity GUI 600 including a graph 602 showing patient activity data accumulated over a period of time t. In the example shown, the activity level is normalized to a 0 to 4 scale (y-axis), and the patient's current activity level 604 is indicated next to the graph 602. The patient activity GUI 600 also includes an alarm button 608 and a help button 610. The functionality of the alarm button 608 is explained hereinafter with reference to FIGS. 6E to 6F, and the functionality of the help button 610 is explained hereinafter with reference to FIG. 6B.

Referring to FIG. 6B, there is show a help GUI 612 displayed via actuation of help button 610. The help GUI 612 provides an explanation of the different numerical activity levels on a scale from 0 to 4, optionally including examples of the types of patient activity corresponding to each activity level, to help contextualize the patient activity information.

The help GUI 612 is organized into a tabular format with five motion activity levels 614 (not separately numbered), ranked from 0 to 4, indicating the increasing level of a patient activity. Each of the five motion activity levels 614 is associated with respective textual explanations 616 (not separately numbered).

Activity level 0 is labeled “Absence of muscle activity” and may indicate a patient is potentially deceased, shows no significant motion or is not in bed.

Activity level 1 is labeled “Static resting intensity” and may indicate that a patient is in calm state, sleep state, is breathing, sedated or in a coma.

Activity level 2 is labeled “Calm and low intensity” and may indicate that the patient is gently scratching itself, hyperventilating, clearing its throat, performing respiration exercises or talking gently.

Activity level 3 is labeled “Medium intensity sustained” and may indicate that the patient is performing myoclonic movements, turning, coughing, modestly exercising, or talking with hand gestures.

Activity level 4 is labeled “Moderate to Intense” and may indicate that the patient is showing convulsions, has experienced a fall, shows aggressions, excessive shaking, is rapidly turning, or has rapidly entered or exited the bed.

A disclaimer (not numbered) at the bottom of the help GUI 612 emphasizes that the examples provided are guidelines and may vary per patient. An OK button 618 may be used to acknowledge and exit the help GUI 612.

Referring to FIG. 6C, there is shown an alarm GUI 620. The user may be given an option to set one or more alarm or notification conditions based on the patient activity level, for example if the activity level is above or below a set threshold for a specified period of time. In the illustrated example, a first alarm 622 is set if a first activity level 624 is equal to 4 for a first time period 626 equal to or greater than 1 second, and a second alarm 628 is set if a second activity level 630 is below 0.9 for a second time period 632 equal to or below 10 minutes. The alarm GUI 620 comprises a sound button 637 which enables adjusting the sound level and a remote alarm button 638 which enables to set up alarms on remote computing devices. If the alarm or notification condition is satisfied, the appropriate medical staff may be notified, for example via the hospital network or a nurse call system, or a record of the activity level may be stored for future reference. An OK button 618 may be used to acknowledge and exit the alarm GUI 620.

Referring to FIG. 6D, there is shown a threshold selection GUI 640 provided for the user to adjust the threshold patient activity level 644 using buttons 642 and 646 for the one or more alarms. An OK button 636 may be used to acknowledge and exit the threshold selection GUI 640.

Referring to FIG. 6E, there is shown a condition selection GUI 650 provided for the user to adjust the type of alert for the one or more alarm, for example whether the alert is triggered by patient activity level being above or equal to the threshold 652 or below the threshold 654. An OK button 656 may be used to acknowledge and exit the condition selection GUI 650.

Referring to FIG. 6F, there is shown a duration selection GUI 660 provided for the user to adjust the threshold duration 662 for the one or more alarms via buttons 664 and 666, for example by selecting if the threshold duration refers to number of seconds via seconds button 668, a number of minutes via minutes button 669, or a number of hours via hours button 670.

An OK button 671 may be used to acknowledge and exit the duration selection GUI 660.

Thus, it should be understood that hospital staff may be able to adjust the alarm conditions based on a medical condition or predicted activity level of the particular patient using computing devices.

In one or more other implementations, the predicted activity level may be used to notify computing devices of caregivers.

Referring to FIG. 6G, there is shown a histogram GUI 672 depicting patient activity data 672 accumulated over a period of time displayed in a histogram format. In the example shown, each bar represents the activity level normalized to a 0 to 4 scale, and the patient's cumulative time spent within each activity level is indicated. The bars are labeled with numerical values indicating the percentage of time the patient spent at each motion level during the specified time frame. The user may be given an interface to view the data in this format over several different durations by selecting a given duration buttons 676 (not separately numbered), which may give an indication of the general activity state of the patient over those durations. This may also provide an indication of how much of the time the patient spent in or out of the hospital bed. Level 0 shows a small percentage of 5.86%, suggesting minimal activity or absence of activity. Level 1 shows a significantly higher value of 30.93%, indicating a greater amount of time spent with low motion intensity. Level 2 has the highest percentage with 36.56%, reflecting the most frequent level of motion. Levels 3 and 4 show decreasing percentages 25.40% and 1.25% respectively, indicating less frequent occurrences of higher motion levels. The histogram GUI 672 comprises a close button 678 to exit the report.

Referring generally to FIGS. 7A to 7F, there is shown complementary information with regard to the patient activity level displayed via a patient movement history GUI 700 to assist a user in interpreting the patient activity level.

Referring to FIG. 7A, the patient movement history GUI 700 includes a center of mass position graph 702 of the current location of the patient's center of mass 704 in the hospital bed, with a gradient representing the transition of the patient's center of mass over the last 2 hours to now. The patient movement history GUI 700 includes navigation controls 705 such as play, pause, stop, and skip, (not numbered) which enables reviewing the patient's center of mass position graph 702 at different moments in time.

The patient movement history GUI 700 includes historical information GUI 706 relevant to the patient's detected activity level in the previous 48 hours with timelines of an indication of the back angle of the hospital bed 708, a timeline of the angle of the bed 710, a timeline of the height of the bed 712, and a timeline indicating whether the patient has exited the bed 714. Each line within the timelines 708, 710, 712 and 714 indicates a different position or movement activity, with changes in position marked by steps in the lines.

Referring to FIG. 7B, the center of mass position graph 702 may be viewed as consecutive images or as a video using navigation controls 714, to allow the user to view the patient's movement within the hospital bed during an interval for which such information was recorded. The center of mass 722 or some other portion of the display image may be grayscale coded or color coded with a gradient based on the patient's activity level, such that the user can observe the patient's activity level over time.

Referring to FIG. 7C, the historical position of the patient's center of mass is displayed as a line or curve 716 indicating the movement over time from an initial location 718 to a final location 719. Segments of the curve 716 may be grayscale coded, color coded or otherwise modified to indicate an activity level of the patient during the corresponding portion of the movement.

Referring to FIG. 7D, another example of displaying the historical position of the patient's center of mass is shown. Curves 720, indicating the movement of the patient's center of mass, represent a span of eight hours of activity that was recorded overnight in this example. It can be seen that the patient awoke twice during the night, as shown by the two separate curves 722 and 724. It can additionally be seen that the patient exited the bed at 726 and returned to the bed at 728. Each of the portions of the curves 722 and 724 may be grayscale coded or color coded to indicate the patient's activity level during that portion of the movement.

Referring to FIG. 7E, graphs 750, 752, 754 and 756 show the position of the center of mass as a point 736 representing the location of the center of mass at particular points in time (i.e., at 20:15, 22:18, 22:24, and 22:31). The position of the center of mass at different times can be seen by viewing the video, animation, or images at different points in time using navigation controls 714. The point 736, or any other suitable portion of the view, can optionally be grayscale coded, color coded or otherwise modified to indicate the patient activity level.

Referring to FIG. 7F, graphs 760, 762, 764 and 766 show the position of the center of mass as a segment of a curve 768 representing the movement of the center of mass over a predetermined period of time. Different portions of the movement of the center of mass can be seen by viewing or animating the video at different points in time, i.e., at 20:15, 22:18 and 22:24. The curve 768, or any other suitable portion of the view, can optionally be grayscale coded, color coded or otherwise modified to indicate the patient activity level.

With reference to FIG. 8, there is shown a computing device 804 connected to one or more load cells 820 and to one or more display devices 830 in accordance with one or more non-limiting implementations of the present technology.

In one or more implementations, the components of the computing device 840 may be similar to the components of the controller 180 of the bed.

The computing device 804 comprises one or more processors 810, one or more memories 812, one or more communication interfaces 814, and input/output interfaces 816. It will be appreciated that the controller 180 is an implementation of a computing device.

In one or more implementations, the one or more processors 810, which may also be referred to as one or more processing devices or one or more processing units, may include a single-core microprocessor. In one or more other implementations, the one or more processors 810 may include a multi-core microprocessor. In one or more alternative implementations, the one or more processors may include one or more of: a microcontroller, a digital signal processor (DSP), an integrated circuit purposed for specific operations within an embedded system, a system on a chip (SoC), a field-programmable gate array (FPGA), and an application-specific integrated circuit (ASIC) configured to carry out the processing and functionalities described herein.

The one or more memories 812 may include volatile and non-volatile memories. In one or more implementations, the one or more memories 812 may include volatile memory, such as random-access memory (RAM), and/or alternatively static random access memory (SRAM) or dynamic random access memory (DRAM). In one or more implementations, the one or more memories 812 may include non-volatile memory, such as flash memory and/or alternatively electrically erasable programmable read-only memory (EEPROM) or ferroelectric RAM (FRAM). The one or more memories 812 are configured to store computer-readable instructions executable by the one or more processors 812 to carry out the processing and functionalities described herein.

The one or more communication interfaces 814 may include wired and wireless communication interfaces to connect the components of the hospital bed 100 to other medical devices, computing devices (e.g., nurse station computer, server(s) and mobile devices), and communication networks (e.g., hospital network or cellular network) to transmit and receive data.

The input/output interfaces 816 may include wired interfaces (e.g. USB, PS/2, RJ45, DB37, serial ports (e.g., RS-232, RS-485), VGA or HDMI ports, power interfaces (e.g., AC power connector and DC power jacks) and data bus interfaces (e.g. CAN Bus) to connect to different components of the hospital bed 100.

The computing device 804 is operatively connected to one or more weight load cells 820 to receive data therefrom. It will be appreciated that the connection between the computing device 804 and the one or more load cells 820 may be wired or wireless.

The computing device 804 is operatively connected to one or more displays 830. The one or more displays 830, also referred to as display screen(s), display device(s), display unit(s) or display interface(s), may include one or more of a liquid crystal display (LCD), light emitting diode (LED) display, organic light emitting diode (OLED) display, plasma displays, e-ink (electronic Ink) display, touch screen displays, quantum dot displays, digital light processing (DLP) projector, head-up displays (HUD), virtual reality (VR) headset displays, and augmented reality (AR) displays.

With reference to FIG. 9, there is shown an environment and system 900 comprising inter alia a patient support apparatus 902, a server 920, a nurse station computer 960, and a client device 962 coupled to one or more communication networks 980 via respective communication links 985 (not separately numbered).

The patient support apparatus 902 may be located in a healthcare facility. The patient support apparatus 902 may be for example located within a zone of a room, a hallway, an intensive care unit, an emergency room, an operating room, and the like. The patient support apparatus 902 or other form of patient support apparatus could be used in various locations without departing from the scope of the present technology.

The patient support apparatus 902 comprises a controller 904 similar to the controller 180 of hospital bed 100. The controller 904 may include at least a portion of the components of the computing device 804 of FIG. 8. The patient support apparatus 902 also comprises one or more load cells (not shown) similar to the load cells 280.

In some implementations, the patient support apparatus 902 may communicate with a headwall (not shown) located in a room. The headwall and patient support apparatus 902 may be configured to transmit and/or receive data. Additionally, or alternatively, the patient support apparatus 902 may connect to a hospital network, a nurse call interface, and other devices via a wired or wireless communication link with the headwall.

The server 920 is configured to inter alia: (i) receive data from and transmit data to the patient support apparatus 902; (ii) receive data from and transmit data to the nurse station computer 960; (iii) receive data from and transmit data to the client device 962; (iii) execute the patient activity monitoring functionality 972; and (v) train, execute and provide access to the one or more ML models 974.

The server 920 executes the patient activity monitoring functionality 972. As part of the patient activity monitoring functionality 972, the server 920 is configured to receive measurements from the one or more load cells 806, determine total weight measured by the one or more load cells 806, determine changes in the measurements including the total weight over different periods of time, determine operational characteristics of the changes in weight, and determine, based on the operational characteristics, a level of activity of a patient in the patient support apparatus 902.

In one or more implementations, the server 920 is configured to determine and monitor a position of a center of mass of the patient in the patient support apparatus 902, determine an activity level and type of activity based on the position of the center of mass.

In one or more implementations, the server 920 is configured to execute one or more ML models 974 to determine a current activity level of the patient or predict an activity level of the patient based on data acquired by the load cells of patient support apparatus 902.

The server 920 is configured to process numerical data and transform the numerical data into multiple forms of visual representations. The visual representations may include, but are not limited to, line plots, bar graphs, pie charts, scatter plots, heat maps, histograms, and color-coded diagrams. Additionally, the server 920 is configured to generate visualizations like 2D and 3D graphs, time lapses, interactive plots, and real-time data visualizations. The generated visual data is then displayed as a part of a GUI, which can be accessed by users through computing devices and/or display units such as display device 830 of computing device 802 (i.e., implemented as a controller), the nurse station computer 960, and the client device 962, for example via patient activity monitoring application 964. The patient activity monitoring application 964 enables users to interact with the visual data, as a non-limiting example via functionalities like zooming, panning, and selecting specific data points for a more detailed view, or program the various previously discussed alarms. Non-limiting examples of GUIs generated by the server 920 are shown in FIGS. 4A to 4F, FIGS. 6A to 6D, and FIGS. 7A to 7F.

The server 920 is configured to transmit notifications to via the one or more communication network 980.

In one or more implementations, the server 920 is configured to execute one or more ML models 974. The one or more ML models 974 may also be accessible via patient activity monitoring application 964.

The one or more ML models 974 are configured to determine, based on one or more of the weights detected by the load cells, operational characteristics determined using the weights of the load cells, the center of mass in the patient support apparatus, a level of activity of a given patient in a patient support apparatus.

In one or more implementations, the one or more ML models 974 are configured to determine a level of activity of a given patient from 0 to 4. It will be appreciated that the level of activity may be determined on a different scale.

In some implementations, the one or more ML models 974 are configured to determine if the changes in the weights detected by the load cells is due to movements of the given patient, or if it is due to another factor such as medical personnel moving in proximity to the patient support apparatus. In such implementations, the one or more ML models 974 may be configured to output the possible factor together with the activity level, or filter and correct the activity level due to possible factor influencing the activity level.

In one or more implementations, the one or more ML models 974 are configured to predict a future level of activity of a given patient based on weights detected by the load cells.

The one or more ML models 974 have been trained to determine activity levels of a patient based on weight data detected by the one or more load cells of a patient support apparatus. It should be understood that the training of the one or more ML models 974 may be performed by the server 920, or by another computing device and provided to the server 920 for inference.

In one or more implementations, the one or more ML models 974 are implemented as classification ML models, also referred to as classifiers. The one or more ML models 974 have been trained on activity level data. It will be appreciated that the activity level data may have been labeled by assessors (e.g., medical personnel) observing a given patient for which the activity level has been generated for a given period. In one or more implementations, the label may be one of a plurality of levels of activity (e.g., from 0 to 4).

In some implementations, the label may also include a level of activity and a type of movement (e.g., convulsions, kicks, exit, etc.) associated with the level of activity.

In one or more other implementations, the one or more ML models 974 may include regression models that are configured to output numerical values indicative of the level of activity.

As a non-limiting example, the one or more ML models 974 may be implemented as classification models such as, but not limited to deep neural networks, support vector machines (SVMs), decision trees, random forest, naive Bayes, logistic regression, as well as ensemble methods (e.g., AdaBoost, gradient boosting, and bagging).

In one or more implementations, the one or more ML models 974 are implemented using the TensorFlow Lite software library. In such implementations, the one or more ML models 974 may be executed, as a non-limiting example, by a microcontroller (e.g., controller of hospital bed 100).

In one or more alternative implementations, the one or more ML models 974 may be executed by another computing device such as the controller of the bed 902, the nurse station computer 960 and/or the client device 962.

In some implementations of the present technology, the server 920 may be connected to the communication network 980 via a communication link 985. In alternative implementations of the present technology, the server 920 may be optional.

The implementation of the server 920 is well known to the person skilled in the art of the present technology. The server 920 may be implemented as one or more computing devices and comprise components similar to the computing device 804, e.g., one or more processors (e.g., central processing unit (CPU) and/or graphics processing unit (GPU)), a memory and/or storage unit, input/output interfaces and communication interfaces. In one or more implementations, the server 920 may be implemented as part of a cloud system (not shown).

It will be appreciated that the server 920 may provide the output of one or more processing steps to another computing device for display, confirmation and/or troubleshooting. As a non-limiting example, the server 920 may transmit data including calculated values, results, and machine learning parameters, for processing and/or display on a computing device such as a smart phone, tablet, and the like.

The nurse station computer 960 is a centralized computing system configured to manage and access patient information, coordinate care, and to enable and facilitate communication among healthcare professionals in a healthcare facility. The nurse station computer 960 is configured to store electronic medical records (EMRs), scheduling and tracking patient appointments, integrating with hospital-wide communication and monitoring systems, assisting in medication management, and providing tools for reporting and analytics.

In one or more implementations of the present technology, the nurse station computer 960 is configured to execute the patient activity monitoring application 964. The patient activity monitoring application 964 may be executed as a stand-alone software application, as an application or may be accessible via a browser application (not shown). Non-limiting examples of interfaces of the patient activity monitoring application 964 are shown in FIGS. 4A to 4F, 6A to 6D, and 7A to 7F.

The environment and system 900 comprise one or more client devices 962 (only one shown in FIG. 9).

The client device 962 is associated with one or more users (not shown), such as medical personnel. As such, the client device 962 can sometimes be referred to as a “computing device”, “end user device” or “client computing device”.

As shown in FIG. 9, the client device 962 may be a tablet used by one or more users, such as medical staff. It will be appreciated that the client device 962 may be implemented as a server, a desktop computer, a laptop, a smartphone, a tablet, and the like without departing from the scope of the present technology.

The client device 962 is configured to execute patient activity monitoring application 964.

In one or more implementations, the client device 962 is configured to access patient activity monitoring application 964 via a browser application (not shown). How the given browser application is implemented is not particularly limited. Non-limiting examples of the given browser application that is executable by the client device 962 include GOOGLE Chrome™, MOZILLA Firefox™, MICROSOFT Edge™, and APPLE Safari™.

Additionally, the environment and system 900 may comprise one or more medical devices and/or computing devices (e.g., mobile device such as a phone, tablet, etc.) connected to the one or more communication networks 980 or directly to components of the environment and system 900.

The one or more communication networks 980 may include one or more of wireless local area networks (WLANs), wired local area networks (LANs), personal area networks (PANs), nurse call systems, wide area networks (WANs), cellular networks, internet of things (IoT) networks, mesh networks, virtual private networks (VPNs), optical fiber networks, and digital health platforms.

How a given communication link 985 (not separately numbered) between the patient support apparatus 902, the server 920, the nurse station computer 960, and the client device 962 is implemented will depend inter alia on how each computing device is implemented. It will be appreciated that each given communication link 985 may be of a different type (e.g., wired or wireless) and may form or connected to a different type of network of the one or more communication networks 980.

Referring to FIG. 10, a flowchart of a method 1000 for determining presence of external motion which may influence the determined level of activity is shown in accordance with one or more non-limiting embodiments of the present technology.

The purpose of the method 1000 is to discriminate between: (i) external motions that may cause fluctuations in the determined level of activity for a given patient; and (ii) internal motions (i.e., caused by the given patient) or absence of external motion on the patient support apparatus which should not influence the determined level of activity. By executing the method 1000, an indication of the presence of external motion may be displayed together with the level of activity to help a medical professional in making decisions with regard to provision of care to the patient.

In some implementations, an indication of absence of external motion detected may be displayed together with the determined level of activity.

The external motions may be due to various factors such as one or more of: functions of the patient support apparatus being activated (e.g., moving the bed, moving the backrest, changing a height of the bed, etc.), another human (e.g., patient, visitor, personnel) leaning, sitting or laying on the bed, integration of equipment to the patient support apparatus, addition or removal of objects (e.g., blankets, pillows, medical devices, trays, personal belongings), medical care or intervention (e.g., turning the patient, cleaning the patient and any type of medical intervention), environmental factors (e.g., accumulation of water, fall of a object) and a technical malfunction/system calibration.

In some implementations, activation of functions of the patient support apparatus may be filtered as causes of external motion by the one or more processors operatively connected to the subsystems of the patient support apparatus. Upon activation of a functionality of the patient support apparatus by a user (e.g., an adjustment to the bed's configuration or the triggering of an integrated feature), the processor may be configured to receive signals from the subsystems of the patient support apparatus and to filter the resultant motions, such that the motions are not taken into account in the determination of the levels of activity.

The internal motions or the absence of motion may generally be attributed to the patient occupying the bed.

In one or more implementations, the method 1000 may be stored in the form of computer-readable instructions within one or more non-transitory storage mediums. The computer-readable instructions, upon being loaded and executed by one or more processors, cause the execution of the method 1000.

In one or more implementations, the method 1000 is executed by one or more processors operatively connected to the one or more load cells (e.g., one or more processors 810 operatively connected to the one or more load cells 820 of FIG. 8). In one or more other implementations, the method 1000 may be executed by a plurality of processors. Non-limiting examples of computing devices and environments and systems for executing the method 1000 are provided in FIG. 8 and FIG. 9.

It should be understood that the method 1000 may be integrated into the method 300 and method 500.

The method 1000 begins at processing step 1002.

At processing step 1002, sensor data is collected and loaded from the load cells 260. The sensor data may include a plurality of readings taken by each of the load cells 260 at regular intervals. It is contemplated that the sensor data may take other forms, such as a total weight detected by the load cells 260 at each time interval, or indications of changes in weight relative to a previous time interval. The sensor data is transmitted from the load cells 260 and received by one or more processors (e.g., one or more processors of the controller 180 of the bed 100 or of another computing device).

At processing step 1004, a total weight detected by the plurality of load cells 260 is determined. The total weight is determined for each time interval at which sensor readings have been acquired by the load cells 260. This determination may be performed concurrently with receiving the sensor data at processing step 1002, for example if the total weight was received from the load cells 260 at processing step 1002, or if there is only a single load cell 260. In some implementations, the total weight may be determined by a processing component of the plurality of load cells 260, the controller 180 of the bed or another computing device.

The method 1000 may optionally proceed to processing steps 1006 or 1008 or may proceed directly to processing step 1010.

At processing step 1006, the one or more processors filter the total weight detected by the load cells to obtain a filtered total weight.

In one or more implementations, the total weight is filtered by applying a low pass infinite impulse response (IIR) filter.

It will be appreciated that the IIR filter is a recursive filter in that the output from the filter is computed by using the current and previous inputs and previous outputs. As a non-limiting example, the IIR filter may be applied by using the following parameters Fc=0.25 Hz, Fs=60 Hz, a0=1.0, a1=−2.94764162, a2=2.89664497, a3=−0.94898587, b0=2.18534588e−06, b1=6.55603764e−06, b2=6.55603764e−06, and b3=2.18534588e−06.

In one or more other implementations, the sensor data may be filtered using techniques such as noise reduction (e.g., a low pass filter), signal smoothing, baseline drift corrections, artifact rejection and the like.

In some implementations, the filtering may be applied using a dynamic sliding window. It will be appreciated that the dynamic filtering with a sliding window could be applied when there is an absence of motion observed in the patient in the bed but the determined level of activity varies slightly (e.g., from 3.1 to 3.2 to 3.1 to 2.9, etc.) due to various factors, however motion is detected, the dynamic window may change to detect the variations in movement which may influence the level of activity. Parameters of the dynamic sliding window may be determined by operators of the present technology.

It will be appreciated that in alternative implementations, the filtering may be optional.

At processing step 1008, a change in the total weight is determined. It should be understood that execution of processing step 1008 is optional depending on implementations of the present technology. The processing step 1008 may be similar to processing step 306 of method 300 of FIG. 3.

At processing step 1010, the one or more processors determine a further operational characteristic.

In implementations where the processing step 1008 is not executed, the one or more processors determine the further operational characteristic based on the filtered total weight (if processing step 1006 is executed) or the total weight (if processing step 1006 is not executed). In such implementations, the further operational characteristic is of a different type than the operational characteristic used for determining the activity level of the patient. The different type of the further operational characteristic may be for example a different function than the function used to calculate the operational characteristic, or a different period of time than the period of time used to calculate the operational characteristic (i.e., equal to or less than the period of time used to calculate the operational characteristic).

As a non-limiting example, the further operational characteristic may be a variance over 60 points (e.g., corresponding to 60 milliseconds (ms)).

In implementations where the processing step 1008 is executed, the one or more processors determine the further operational characteristic based on the changes in total weight, similar to processing step 308 of method 300 of FIG. 3, and the further operational characteristic may be of the same type as the operational characteristic used for determining the activity level of the patient. In such implementations, the further operational characteristic and the operational characteristic may be any suitable function of at least one of the magnitudes of the changes in total weight or the time duration of the changes in total weight, such as a variance or a range of the weight over a period of time. The operational characteristic may also include or be based on a rate of change of weight, a standard deviation, a weight change frequency, a cumulative weight change, and a moving average.

At processing step 1012, the one or more processors compare the further operational characteristics with a threshold.

In one or more implementations, the further operational characteristic is the variance of the filtered total weight. As a non-limiting example, a value of the threshold of the variance may be of 100,000.

In one or more implementations, the threshold may be a threshold based on the given patient characteristics such as the weight of the given patient calculated prior to executing processing step 1012.

It will be appreciated that using a threshold based on the weight of the given patient contributes to discriminating between changes in activity level and/or variations in the values of the operational characteristic being caused either by the patient occupying the bed or by an external source. As a non-limiting example, setting a threshold based on the weight of the patient occupying the bed facilitates accounting for scenarios where external motion results in minor weight variations relative to the patient's weight, which may be filtered out by the one or more processors. For instance, an object weighing 1 kg falling onto a bed with a patient weighing over 200 kg may be filtered out or considered to be an absence of external motion or as an internal motion of the patient. Conversely, if the bed is occupied by a smaller patient weighing 50 kg, the same 1 kg object may produce a comparatively more noticeable change in the load cell readings and may be detected. Thus, the threshold based on the weight of the given patient enables to reach a similar conclusion for patient of different weights, i.e., presence of an external motion

At processing step 1014, if the further operational characteristic is above the threshold, the one or more processors determines that there is presence of external motion.

The external motion may be caused by one or more of: another human (e.g., patient, visitor, personnel) being in contact with the patient support apparatus (e.g., leaning, sitting or laying on the bed), the integration of equipment to the patient support apparatus, activation of functions of the patient support apparatus, addition of objects (e.g., blankets, pillows, medical devices, trays, personal belongings), a medical intervention, environmental factors (e.g., accumulation of water, fall of an object) and a technical malfunction/system calibration.

In one or more implementations, an indication of the presence of external motion may be output together with the level of activity (e.g., processing step 310 of method 300) and displayed (e.g., processing step 312 of method 300) on one or more display devices.

Non-limiting examples of indicators of presence of external motion are shown in FIGS. 4A, 4B and 4F as indicators 408A, 408B, and 408F in the graphs 400A, 400B, and 400C, respectively, which correspond to the backrest of the bed being adjusted (e.g., raised), the patient leaving the bed and a person walking near the hospital bed, respectively.

At processing step 1016, if the variance is below the threshold, the one or more processors determine that there is one of: a presence of internal motion and absence of motion of the given patient. It will be appreciated that the determination at processing step 1016 provides confidence that the degree of patient activity that is determined (e.g., at processing step 310 of FIG. 3) is likely not due to external factors or motions.

Non-limiting examples of indicators of absence of internal motion are shown in FIGS. 4C, 4D and 4E as indicators 408C, 408D, and 408E in graphs 400C, 400D, and 400E respectively, which correspond to a patient kicking the bed, a patient having convulsions and a patient moving his hands occasionally, respectively.

In one or more implementations, the determination of the presence of external motion or the determination of the presence of internal motion/absence of motion is indicative of a level of confidence in the level of activity of the patient determined by executing implementations of the methods 300 and 500.

With reference to FIG. 11, there is shown an example of a home interface GUI 1100 which may be displayed on a display device such as the one or more display devices 830 (FIG. 8) associated with a patient support apparatus (e.g., hospital bed 100 of FIG. 1 or patient support apparatus 902 of FIG. 9). It will be appreciated that in alternative implementations, the home interface GUI 1100 may also be displayed on another computing device such as the nurse station computer 960 or the client device 962.

The home GUI 1100 enables inter alia to display different information related to the status of the bed or of the given patient including information determined by using the load cells of the hospital bed.

The home GUI 1100 includes a time in bed section 1102, a bed information section 1110, an arm detection button 1120, a scale button 1130, a patient risk management button 1134 and preference button 1140.

The home interface GUI 1100 also includes quick link buttons with an in-bed time quick link button 1150 which enables quickly accessing the in-bed time GUI 1300 (FIG. 13), a bed status quick link button 1152 to access the bed status in the home GUI 1100 and a fall risk quick link button 1154 to notify of a fall risk of the patient.

The time in bed section 1102 shows an icon with values of a ratio of the time spent in bed by the patient to total time recorded (2 h35/4 h) with a corresponding percentage of the time spent in bed (65%) and an icon with the number of bed exits 1106 by the patient (3), as detected by using the load cells of the bed (e.g., load cells 260 of bed 100 of FIG. 1).

The bed information section 1110 shows a diagram of the bed 1112 with the current configuration of the bed, with values of the width of the bed (41″), a backrest icon 1114 depicting an angle of the backrest with the associated value (38 degrees), a height icon 1116 indicating height of the bed with the associated height value (10″), and change in positions icon 1118 with a value indicating an angular inclination of the bed in the Trendelenburg or reverse Trendelenburg positions (10).

The user may click or select the arm detection button 1120, the scale button 1130, the patient risk management button 1134 and the preference button 1140 to display respectively an arm detection GUI (not shown), a scale GUI (not shown), a patient risk management GUI 1200 (shown in FIG. 12) and a preferences GUI (not shown).

With reference to FIG. 12, there is shown the patient risk management GUI 1200. The patient risk management GUI 1200 includes a bed exit button 1202, an in-bed time button 1204, an inform button 1206 and view log button 1208.

The user may click or select the bed exit button 1202, the in-bed time button 1204, the inform button 1206 and the view log button 1208 to display respectively a bed-exit GUI (not shown), an information GUI (not shown), the in-bed time GUI 1300 (FIG. 13) and the in-bed time events GUI (FIG. 14).

With reference to FIG. 13, there is shown the in-bed time GUI 1300.

The in-bed time GUI 1300 includes a current status section 1370 with an icon showing values for the time spent in bed (23 min), an available history section 1372 showing the history available for time in bed since the bed was unplugged from the hospital power grid (1 h30), a bed time ratio section 1374 with values showing time in bed with a percentage if time in bed (54 minutes/60%) and bed exit section 1376 depicting an icon of bed exit with a number of exits from the bed by the patient (3 exits).

The in-bed time GUI 1300 includes a plurality of duration buttons 1302, 1304, 1306, and 1308 corresponding to durations of 4 hours (1302), 8 hours (1304), 12 h (1306) and a reset duration button (1308). By selecting or clicking the buttons 1302, 1304 and 1306, the user may cause calculation and display of the different values for the selected duration (i.e., the last 4 hours if button 1302 is selected, the last 8 hours if button 1304 is selected, and the last 12 hours if button 1306 is selected) in the current status section 1370, the available history section 1372, the time ratio section 1374 and the bed exit section 1376. In use, one of the duration buttons 1302, 1304, 1306 may be highlighted when the information associated therewith is displayed in the sections 1370, 1372, 1374, 1376. The different time periods associated with the duration buttons 1302, 1304, 1306 may be different in other implementations. The reset duration button 1308 enables resetting the duration (i.e., starting the calculation of the values associated with the current status section 1370, the available history section 1372, the time ratio section 1374 and the bed exit section 1376 from zero).

The in-bed time GUI 1300 includes an event button 1360 to show an in-bed time events GUI 1400 (FIG. 14), a show in home button 1362 which enables showing information from the in-bed time GUI 1300 in the home GUI 1100, a show in inform button 1364 which enables showing information from the in-bed time GUI 1300 in an information GUI (not shown), and a close button 1366 which enables exiting the in-bed time GUI 1300.

The in-bed time GUI 1300 also includes quick link buttons with an in-bed time quick link button 1350 which enables quickly accessing the in-bed time GUI 1300, a bed status quick link button 1352 to access the bed status in the home GUI 1100 and a fall risk quick link button 1354 to notify of a fall risk of the patient.

It should be noted that when the bed is unplugged from the hospital power grid, events indicative of a level of activity may not be recorded or may be recorded at lower frequencies to save power, for example if the bed is powered by a battery (not shown). Thus, historical information regarding the events or the level of activity may not be available or may be provided at lower frequencies with an indicator showing that the information may not be reliable due to the bed being unplugged from the electrical power grid.

With reference to FIG. 14, there is shown the in-bed time events GUI 1400. The events GUI 1400 includes a log of events displayed line by line.

The in-bed time events GUI 1400 includes a header indicating the In-Bed Time Events followed by a timestamp (not numbered) of the current date and time.

The in-bed time events GUI 1400 includes a log of events 1402 displayed as a list of events with corresponding icons, where each event is associated with a timestamp indicating the occurrence of the event. The log of events 1402 shows the recent events in descending order. It will be appreciated that the log of events 1402 may display the events in a different order without departing from the scope of the present technology.

The in-bed time events GUI 1400 includes duration buttons 1420, 1422, and 1424 which enables to display the log of events 1402 for different durations (corresponding respectively to durations of 4 h, 8 h, and 12 h). The 12 h duration button 1424 is selected to display the log of events 1402 in FIG. 14 is displayed for the duration of 12 h.

In the example shown in FIG. 14, the log of events 1402 includes a power restored event 1410 with a timestamp of June 6^{th} at 11:38, with an icon and text indicating when the bed was connected to the electrical power grid of the facility. The log of events 1402 includes a bed without power event 1412 with an icon and text indicating the duration the bed was without power for 26 min. The log of events 1402 includes a power lost event 1414 with an icon and text indicating power was lost with a timestamp of June 6^{th} at 11:12. The log of events 1402 includes a time in bed event 1416 showing an icon and information including a percentage of time spent in bed (84%) with a corresponding time ratio (8 h39/12 h) and a number of exits with a timestamp of June 6^{th} at 11:10. The log of events 1402 may alternatively or additionally show time in bed events (such as time in bed event 1416) at different durations (e.g., every 4 hours or every 8 hours).

The in-bed time events GUI 1400 includes navigation controls 1404 and 1406 to navigate between other pages of the log of events 1402 with a text 1408 showing the current page and the total number of pages of events recorded.

The in-bed time events GUI 1400 includes a close button 1430 which enables exiting the in-bed time events GUI 1400.

Using any of the above methods, alone or in combination with other information from the load cells or other sensors disposed on or near a patient support apparatus, it may be possible to distinguish different types of patient activity or events. As a non-limiting example, it may in some cases be possible to distinguish patient activities such as speech, seizures, rolling over in bed, and exiting/entering the bed. In another non-limiting example, a patient rolling over in his sleep may be distinguished from a kick by a movement of the patient's center of mass concurrently with the detected activity level. It might also be possible to alert medical staff as to what type of activity is most likely occurring, in addition to indicating the patient's activity level. This may assist medical staff in responding to patients with an appropriate level of urgency.

Using any of the above methods, alone or in combination with other information from the load cells or other sensors disposed on or near the patient support apparatus, it may be possible to predict future patient activity, or to predict a future medical condition or medical risk associated with the patient. Using this information, medical staff may be able to engage in preventative interventions to reduce the probability of a future medical condition occurring. For example, a patient who has become agitated in the past can be monitored for activity that precedes the agitation event, so that future agitation events can be predicted (for example by one or more artificial intelligence (AI) models such as the one or more ML models 974) and handled appropriately.

In another non-limiting example, if a patient is required to limit physical activity, or to engage in a minimum amount of physical activity to aid in recovery, monitoring the patient's activity level may allow an AI model or medical staff to predict that the patient is not likely to achieve the target activity level for the day, and medical staff could then intervene appropriately. Medical staff may also monitor the time a patient rests in bed and recommend more frequent bed exits to ease recovery. In another non-limiting example, a patient who has had particularly low levels of activity may be judged to be at a higher risk for developing bedsores, and medical staff could then intervene appropriately to prevent this outcome.

In addition, if an activity event has been logged or recorded in the past, it may be possible to use information about the activity event, such as its amplitude and duration, in combination with information gathered by other sensors around the same time, to determine the nature of the event after the fact.

It should be understood that one or more of the hospital bed or the computing device described herein are equipped with the necessary components to carry out the functions described above, such as one or more screens or displays, one or more computer processors, and one or more non-transitory memories connected to the processors and containing executable instructions for causing the processor to carry out the functions described above.

With reference to FIGS. 15 to 22, techniques for determining immobility of a patient will be described. Immobility of a patient may be determined by using embodiments of the methods and systems for determining a patient activity level described herein. One or more of the present embodiments enable obtaining a cumulative and less reactive measure of patient activity agitation to determine if a patient is immobile. This would help prevent pressure sores and promote mobility via appropriate care by personnel.

More specifically, the immobility of a patient may be determined by determining that the patient activity level is below a threshold, and by determining that the movement of the center of gravity (center of mass) is below a movement threshold, which may indicate that a patient is immobile.

It will be appreciated that patient immobility may be indicative of a risk of a patient developing bed sores, particularly when the patient has been immobile for a long period of time and depending on other patient health factors. Thus, the indication of patient immobility may be transferred to remote computing devices (e.g., smartphones, tablets, computers, nurse station computers) to notify appropriate care personnel to attend the patient. Additionally or alternatively, the indication of patient immobility may also be used to trigger functions of a patient support apparatus or mattress based on the patient level activity being below immobility thresholds and durations.

FIG. 15 illustrates a flow chart of a method 1500 for determining immobility of a patient in a patient support apparatus in accordance with one or more non-limiting embodiments of the present technology.

The method 1500 may be executed as part of method 300 of FIG. 3, and it should be noted that processing steps 1502-1504 are similar to processing steps 302-304. In some embodiments, method 1500 is executed every time method 300 is executed. In one or more other embodiments, the method 1500 is executed if the functionality of determining immobility has been activated, for example via a user interface element associated with the patient support apparatus (e.g., via a control interface of the bed or a menu in a remote computing device).

The method 1500 may be stored in the form of computer-readable instructions in at least one non-transitory storage medium operatively connected to the at least one processor. The at least one processor, upon executing the computer-readable instructions, is configured to execute method 1500. In one or more embodiments, the at least one processor may be a processing device of the patient support apparatus (e.g., hospital bed 100). In one or more other embodiments, the at least one processor may be directly or indirectly connected via a wired or wireless connection to the at least one load sensor and controller of the hospital bed. It will be appreciated that the execution of the method 1500 may also be distributed among a plurality of computing devices.

According to processing step 1502, the at least one processor receives sensor data. In one or more embodiments, the at least one processor receives sensor data from the load cells 260 of the hospital bed 100. As a non-limiting example, data may be received from each of the four load cells at time intervals of 20 ms.

According to processing step 1510, the at least one processor computes a level of patient activity.

The at least one processor may execute different embodiments of processing steps 304-310 of method 300 of FIG. 3 to compute the level of patient activity, i.e., calculate total weight from the received sensor data, calculate changes in total weight, calculate an operational characteristic, and compute a level of patient activity based on the operational characteristic. It will be appreciated that the operational characteristic may be any suitable function of at least one of the magnitudes of the changes in total weight or the time duration of the changes in total weight, such as a variance or a range of the weight over a period of time. As a non-limiting example, the level of patient activity may be determined based on a variance of the weight changes and the total weight detected by the load cells.

According to processing step 1512, the level of patient activity is compared to a threshold. The threshold may correspond to an immobility threshold.

It will be appreciated that as part of method 1500, the level of patient activity may be compared to a threshold continuously or at given time intervals when executing the method 300.

In one or more embodiments, the threshold may be a predetermined threshold. As a non-limiting example, the threshold may be set to 2.5 (on a scale from 0 to 4). It will be appreciated that other values may be set by operators of the present technology, may be determined experimentally and/or may depend on the scale on which the level of activity is expressed. It is also contemplated that more than one threshold may be used for different levels of immobility.

In one or more alternative embodiments, the threshold may be a dynamic threshold. As a non-limiting example, the threshold may be adjusted dynamically according to various detected parameters or may be patient specific.

It should be noted that when the level of patient activity is computed, the total weight of the patient detected by the load sensors is tracked within a threshold weight such that if the total weight changes above the threshold weight due to an external motion (e.g., additional person or object on the bed), the at least one processor causes transmission of an indication that there is external motion when computing the level of patient activity and determining immobility, which could bias the computed values. This indication enables the appropriate staff to attend to the patient in case the external motion is involuntary. In some embodiments, the appropriate staff may confirm via the user interface that the external motion is caused voluntarily, which may resume the computation of the level of patient activity and/or remove the display of the indication of the external motion.

If the level of activity is below the threshold, the method 1500 advances to processing step 1514.

If the level of activity is above the threshold, the method 1500 may stop or may return to processing step 1502.

According to processing step 1514, the at least one processor computes a movement of the center of gravity of the patient on the patient support apparatus.

In some embodiments, the tracking of the center of gravity is performed only if the activity level is below the immobility threshold.

In one or more embodiments, the at least one processor uses the data received from each of the load cells 260 to calculate the location of the center of gravity of the patient at predetermined frequencies (e.g., every 20 ms) during a period of time. By doing so, the at least one processor determines the movement of the center of gravity of the patient by determining the difference between positions of the computed center of gravity during a given time interval. The movement of the center of gravity may be computed in the lateral direction, the longitudinal direction, or a combination thereof, depending on the configuration and number of load cell sensors. The movement of the center of gravity may also be monitored within an area of the patient support surface of the bed.

According to processing step 1516, the at least one processor determines if the center of gravity of the patient has moved above or below a movement threshold.

It will be appreciated that the movement of the center of gravity is determined and compared to a threshold to confirm that the patient shows minimal agitation and lateral or longitudinal movement which is indicative of immobility of the patient.

According to processing step 1518, if the movement of the center of gravity is below the movement threshold, the at least one processor determines that the patient is immobile.

It will be appreciated that the threshold may be a distance threshold. As a non-limiting example, the distance threshold may be 2.5 cm. The at least one processor may compare the distance between each time point, and compare it to the distance threshold to obtain a comparison result.

In one or more alternative embodiments, the at least one processor may determine the velocity of the center of gravity of the patient, and if the velocity is below a velocity threshold (e.g., 0), the at least one processor may determine that the patient is immobile.

If the movement of the center of gravity is above the movement threshold, the method 1500 may stop or may return to processing step 1502. In one or more embodiments, the method 1500 may continuously compare the movement of the center of gravity to a threshold if the level of patient activity is below the immobility threshold.

According to processing step 1518, the at least one processor determines that the patient is immobile based on the patient activity level being equal to or below the immobility threshold and the movement of the center of gravity being equal or below the movement threshold. It will be appreciated that both conditions have to be satisfied to determine that the patient is immobile.

In one or more embodiments, the at least one processor starts an immobility timer and continues recording and verifying the patient activity level and the movement of the center of gravity to track the time period during which the patient is active and/or immobile.

According to processing step 1520, the at least one processor transmits the indication of immobility of the patient together with the period of immobility, which causes displaying of the indication of immobility.

In one or more embodiments, the at least one processor causes displaying of the indication of immobility on a display associated with the hospital bed 100. As a non-limiting example, the display may be mounted to hospital bed 100.

In one or more embodiments, the at least one processor may transmit the indication of immobility to another computing device, such as a server, a mobile computer device or nurse station computer for displaying the indication of immobility, which may cause the device to display the time during which the patient is immobile in a graphical user interface (GUI).

In one or more embodiments, the indication of immobility of the patient is associated with an immobility time period corresponding to time period during which the patient is immobile. The immobility time period with the mobility level may be displayed on a graphical user interface (GUI). It will be appreciated that the patient activity level, the immobility level and associated time periods may be displayed in various forms, such as in timeline form. The patient activity level and immobility level with associated time periods may be displayed in a dedicated area on the home screen GUI such as a banner, on a pop-up, or on a dedicated GUI. It should be understood that the indication of immobility may be displayed in various forms on one or more displays, depending on the configuration. The indication of immobility may be displayed in binary form (e.g., ‘YES’ or ‘NO’), in text form, in graphical form, and optionally with the time during which the patient has been immobile.

In some embodiments, if the immobility time is above a threshold, the at least one processor may transmit or cause generation of an alert, which may be visual, audio, physical or a combination thereof. It will be appreciated that this may be useful to notify the appropriate staff that the patient has been immobile, and that the patient should be attended to, as the patient may be at risk of developing bed sores.

As a non-limiting example, if the immobility time is above a threshold, the at least one processor may cause execution of an alarm on displays screens associated with the patient activity level, such as the display screen of the control panel of the bed, the nurse station computer, and remote computing devices, as well as flashing lights associated with the patient support apparatus (e.g., bumper lights, room lights, hallway lights, etc.). The alert may be stopped by medical personnel having attended the patient.

In one or more alternative embodiments, if the immobility time is above a threshold, the at least one processor may cause execution of functions of the patient support apparatus and/or the patient support surface (e.g., mattress), such as, but not limited to, automated repositioning, lateral tilting or rotation, pressure redistribution, microclimate management, and the like to prevent the patient from developing bed sores until intervention by a medical practitioner.

FIG. 16A to FIG. 16D show graphs 1605A, 1605B, 1605C, 1605D of different levels of force detected by each of the four load cells of the bed, where data sensed by each load cell corresponds to a respective curve on the graph.

In FIG. 16A, graph 1605A shows force measurements of four load cells 1610A, 1612A, 1614A, 1616A with stable measurements with minimal variations which correspond to levels of patient activity between 0 to 1 on a scale of 0 to 5.

In FIG. 16B, graph 1605B shows force measurements of four load cells 1610B, 1612B, 1614B, 1616B with small variations in force measurement, which corresponds to levels of patient activity between 1 to 2 on a scale of 0 to 5.

In FIG. 16C, graph 1605C shows force measurements of four load cells 1610B, 1612B, 1614B, 1616B with higher force variations, which corresponds to levels of patient activity between 2 and 3 on a scale of 0 to 5.

In FIG. 16D, graph 1605D shows force measurements of four load cells 1610D, 1612D, 1614D, 1616D with significant force variation, which corresponds to levels of patient activity between 3 to 5 on a scale of 0 to 5.

With reference to FIG. 17, there is shown a patient activity graph 1700 of the patient activity level on the y-axis, which ranges from 0 to 5, with time on the x-axis ranging from 0.00 to 10.00 minutes.

In patient activity graph 1700, between 0 and 2 minutes, the patient's activity begins at a low level and gradually increases, reaching a peak slightly above 2 on the y-axis. After this peak, the activity decreases slightly but remains above 1. From 2 to 4 minutes, the activity fluctuates within a moderate range, initially dipping before gradually increasing and peaking close to 3, followed by a slight decline. In the 4 to 6-minute interval, the activity level rises steadily, reaching a higher peak around 5.5 minutes, where it briefly stabilizes before dropping sharply toward the end of this period. Between 6 and 8 seconds, the activity remains high initially but then experiences a sharp decline, reaching its lowest point around 7.5 minutes, where it approaches a value close to 1. From 8 to 10 minutes, the activity begins to rise again after reaching its lowest point, showing a gradual increase toward the end of the graph.

Thus, in the patient activity graph 1700, the patient shows immobility under a patient activity level of 2.5, which is between approximately 0 to 1.5 minutes, between approximately 2.5 to 3.5 minutes, and between approximately 7 to 9.5 minutes.

Reference is now made to FIGS. 18 to 22, which illustrate different graphical user interfaces associated with patient immobility. In FIGS. 18 to 22, a patient is considered immobile if the following conditions are met for a certain period: no significant movement detected by the bed, and no significant change in the movement of the center of mass.

FIG. 18 shows a non-limiting example of a patient movement GUI 1800 in accordance with non-limiting embodiments of the present technology.

The patient movement GUI 1800 includes a bed system status section 1810 and an in-bed time section 1820. The bed system status section 1810 shows the status of the various components of the bed and the weight of the patient.

The in-bed time section 1820 shows timelines of patient activity between 16:00 and 01:00, with an immobility timeline 1840, in-bed timeline 1850, and out of bed timeline 1860. The time duration or interval for each of immobility, in-bed time and out of bed time is displayed in each bar. It can be seen that the patient was out of bed between 16:00 and 17:18 for a duration of 2 hours 18 minutes, and was in bed between 17:18 and 01:20 for a duration of 8 hours 2 minutes. The patient has been detected as being immobile for 21 minutes between 18:00 and 19:00, 36 minutes between 19:00 and 20:00, 2 hours 10 minutes between 20:45 and 23:00, and for 55 minutes between 0:25 and 1:20.

FIG. 19 shows another patient movement GUI 1900 similar to the patient movement GUI 1800 of FIG. 18 with an in-bed system status section 1910 and an in-bed time section 1920.

In this example, the in-bed time section 1920 includes a patient activity timeline between 16:00 and 01:00, with a motion level graph 1930, immobility timeline 1940, in-bed timeline 1950, and out of bed timeline 1960, and markers for set alarm time (not numbered), postponed alarm time (not numbered) and confirmed alarm time (not numbered),

In this example, a user has set an alarm time for a given duration at 22:00, and has postponed the alarm around 22:20, and confirmed the alarm around 22:50.

FIG. 20 shows a non-limiting example of a home GUI with immobility 2000, which is similar to a home interface GUI 1100 shown in FIG. 11, and only the differences with home interface GUI 1100 will be described.

In this example, a tracking of patient immobility function has been activated, and the home GUI with immobility 2000 has an immobility section 2010, which shows an immobility time duration of 00:05 hours or 5 minutes, and Max, which corresponds to a maximum duration of patient immobility of 00:45 hours or 45 minutes since their last installation on the bed.

FIG. 21 illustrates an embodiment of an in-bed time GUI 2100, which is similar to the in-bed time GUI 1300 shown in FIG. 13. In this example, a tracking of patient immobility function has been activated.

The in-bed time GUI 2100 includes an immobility section 2160. The immobility section 2160 includes an icon of the patient in bed.

The immobility section 2160 includes an in-bed timer 2162 indicating that the patient has been in bed since 1:05, an immobility timer 2164 indicating that the patient has been immobile for 00:05 hours or 5 minutes, and max duration timer 2166 indicating that the patient has been immobile for a maximum of 00:45 hours or 45 minutes since their last installation on the bed.

FIG. 22 shows an immobility management GUI 2200. The immobility management GUI 2200 includes an immobility notification section 2220 and an alarm section 2230. The immobility notification section 2220 includes a notification button 2222 that can be pressed to be activated or deactivated, and a timer 2224 that can set a threshold time of immobility after which a notification may be emitted. It will be appreciated that the notification may appear in the GUI of a display screen and/or on another computing device connected to the hospital bed 100.

The alarm section 2230 includes an alarm button 2232 that can be pressed to activate or deactivate an alarm, and an alarm timer 2234 to set a threshold time of immobility after which the alarm may be activated (above 120 minutes in the example shown in FIG. 22).

The alarm section 2230 further includes a sound button 2242 that can be used to activate or deactivate an audible alarm, and a remote alarm button 2244 that can be used to activate or deactivate a remote alarm.

It should be expressly understood that not all technical effects mentioned herein need to be enjoyed in each and every implementation of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A system for determining immobility of a patient in a patient support apparatus, the system comprising:
a non-transitory storage medium storing computer-readable instructions;
at least one processor operatively connected to the non-transitory storage medium, the at least one processor being operatively connected to at least one load cell of the patient support apparatus;
the at least one processor, upon executing the computer-readable instructions, being configured for:
receiving, from the at least one load cell, sensor data comprising weight components exerted by the patient;
computing, based on the sensor data, a level of patient activity of the patient without calculating a center of gravity of the patient;
determining if the level of patient activity is below a predetermined threshold during a given period of time;
if the level of patient activity is below the predetermined threshold:
computing a movement of the center of gravity of the patient during the given period of time;
determining if the movement of the center of gravity of the patient is below a movement threshold;
if the movement of the center of gravity is below the movement threshold:
determining that the patient is immobile during the given period of time; and
transmitting, to a user interface operatively connected to the at least one processor, an indication of immobility of the patient during the given period of time.

2. The system of claim 1, wherein the at least one processor is further configured for, prior to said computing the level of patient activity of the patient:
computing, based on the sensor data comprising the weight components, a total weight detected by the at least one load cell;
computing changes in the total weight detected by the at least one load cell over the given period of time;
computing an operational characteristic of the changes in the total weight based on at least one of a magnitude and a time duration of the changes in total weight; and
wherein said computing the level of patient activity of the patient is based on the operational characteristic.

3. The system of claim 2, wherein said computing the level of patient activity of the patient during the given period of time based on the operational characteristic comprises:
computing a variance of the changes in total weight over the given period of time; and
linearizing the variance of the weight to obtain the level of patient activity of the patient.

4. The system of claim 3, wherein said computing the movement of the center of gravity of the patient comprises: computing a difference between consecutive positions of the center of gravity of the patient during the given period of time.

5. The system of claim 4, wherein the user interface comprises a display operatively connected to the at least one processor, and wherein the at least one processor is further configured for displaying the indication of immobility and the given time period on a display operatively connected to the at least one processor.

6. The system of claim 5, wherein the at least one processor is configured for displaying the indication of immobility and the given time period in a timeline.

7. The system of claim 6, wherein the at least one processor is further configured for:
receiving, from the user interface, a maximum time of immobility;
determining if the given period of time is equal to above the maximum time of immobility; and
if the given period of time is equal to or above the maximum time of immobility:
transmitting an indication that the patient has been immobile above the maximum time.

8. The system of claim 7, wherein said transmitting indication that the patient has been immobile above the maximum time causes generation of at least one of: a visual alert and an audio alert.

9. The system of claim 8, wherein the level of patient activity varies between 0 and 4.

10. The system of claim 9, wherein the predetermined threshold is equal to 2.5.

11. A patient support apparatus comprising:
a frame;
at patient support surface supported by the frame;
at least two load cells disposed between the frame and the patient support surface; and
at least one processor operatively connected to the at least one two cells, the at least one processor being configured for:
receiving, from the at least two load cells, sensor data comprising weight components exerted by the patient;
computing, based on the sensor data, a level of patient activity of the patient;
determining if the level of patient activity is below a predetermined threshold during a given period of time;
if the level of patient activity is below the predetermined threshold:
computing a movement of a center of gravity of the patient during the given period of time;
determining if the movement of the center of gravity of the patient is below a movement threshold;
if the movement of the center of gravity is below the movement threshold:
determining that the patient is immobile during the given period of time; and
transmitting, to a user interface operatively connected to the at least one processor, an indication of immobility of the patient during the given period of time.

12. A method for determining immobility of a patient supported by a patient support apparatus, the patient support apparatus having at least one load cell, the method being executed by at least one processor operatively connected to the at least one load cell, the method comprising:
receiving, from the at least one load cell, sensor data comprising weight components exerted by the patient;
computing, based on the sensor data, a level of patient activity of the patient without computing a center of gravity of the patient;
determining if the level of patient activity is below a predetermined threshold during a given period of time;
if the level of patient activity is below the predetermined threshold:
computing a movement of the center of gravity of the patient during the given period of time;
determining if the movement of the center of gravity of the patient is below a movement threshold;
if the movement of the center of gravity is below the movement threshold:
determining that the patient is immobile during the given period of time; and
transmitting, to a user interface operatively connected to the at least one processor, an indication of immobility of the patient during the given period of time.

13. The method of claim 12, further comprising, prior to said computing the level of patient activity of the patient:
computing, based on the sensor data comprising the weight components, a total weight detected by the at least one load cell;
computing changes in the total weight detected by the at least one load cell over the given period of time;
computing an operational characteristic of the changes in the total weight based on at least one of a magnitude and a time duration of the changes in total weight; and
wherein said computing the level of patient activity of the patient is based on the operational characteristic.

14. The method of claim 13, wherein said computing the level of patient activity of the patient during the given period of time based on the operational characteristic comprises:
computing a variance of the changes in total weight over the given period of time; and
linearizing the variance of the weight to obtain the level of patient activity of the patient.

15. The method of claim 14, wherein said computing the movement of the center of gravity of the patient comprises: computing a difference between consecutive positions of the center of gravity of the patient during the given period of time.

16. The method of claim 15, further comprising: displaying the indication of immobility and the given time period on a display operatively connected to the at least one processor.

17. The method of claim 16, further comprising:
receiving, from the user interface, a maximum time of immobility;
determining if the given period of time is above the maximum time of immobility; and
if the given period of time is equal to or above the maximum time of immobility:
transmitting an indication that the patient has been immobile above the maximum time.

18. The method of claim 17, wherein said transmitting indication that the patient has been immobile above the maximum time causes generation of at least one of: a visual alert and an audio alert.

19. The method of claim 11, wherein the level of patient activity varies between 0 and 4.

20. The method of claim 19, wherein the predetermined threshold is equal to 2.5.
